(19) United States
(12) Patent Application Publication
(10) Pub. No.: US 2019/0389972 A1
(43) Pub. Date: Dec. 26, 2019

(54) MODIFIED CK AND CH1 DOMAINS

(71) Applicant: I-MAB, Grand Cayman (KY)

(72) Inventors: Yongqiang Wang, Shanghai (CN); Lei Fang, Shanghai (CN); Zhengyi Wang, Shanghai (CN); Bingshi Guo, Shinghai (CN); Jingwu Zang, Shanghai (CN)

(21) Appl. No.: 16/489,970

(22) PCT Filed: Jan. 15, 2019
(86) PCT No.: PCT/CN2019/071740

§ 371 Date: Aug. 29, 2019

(30) Foreign Application Priority Data
Jan. 15, 2018 (CN) PCT/CN2018/072564

(51) Int. Cl. C07K 16/46

(52) U.S. Cl. CPC C07K 16/46; C07K 2317/94; C07K 2317/50; C07K 2317/522

(57) ABSTRACT

Provided are antibody and antigen-binding fragment with modified Cκ and CH1 domains that still enable pairing of the Cκ and CH1 domains but have reduced pairing compared to wild type CH1 and Cκ domains without the modification. Such modifications can particularly useful for preparing bispecific antibodies which two different pairs of Cκ and CH1 domains.

### BACKGROUND

A bispecific monoclonal antibody (BsMAb, BsAb) is an artificial protein that can simultaneously bind to two different types of antigen or two different epitopes of the same antigen. BsAbs can be manufactured in several structural formats, and current applications have been explored for cancer immunotherapy and drug delivery.

There are many formats of BsAb. An IgG-like BsAb retains the traditional monoclonal antibody (mAb) structure of two Fab arms and one Fc region, except the two Fab sites bind different antigens. The most common types are called trifunctional antibodies, as they have three unique binding sites on the antibody: the two Fab regions, and the Fc region. Each heavy and light chain pair is from a unique mAb. The Fc region made from the two heavy chains forms the third binding site. These BsAbs are often manufactured with the quadroma, or the hybrid hybridoma, method.

However, the quadroma method relies on random chance to form usable BsAbs, and can be inefficient. Another method for manufacturing IgG-like BsAbs is called “knobs into holes,” and relies on introducing a mutation for a large amino acid in the heavy chain from one mAb, and a mutation for a small amino acid in the other mAb's heavy chain. This allows the target heavy chains (and their corresponding light chains) to fit together better, and makes BsAb production more reliable.

While this knob-into-holes approach solves the heavy chain homodimerazation problem, it did not address the issues regarding mispairing between the light chain and heavy chains from two different antibodies. There is a need to provide better BsAbs that are easier to prepare, and have better clinical stability and efficacy.

### SUMMARY

The present disclosure provides antibodies and antigen-binding fragments with modified Cκ and CH1 domains that still enable pairing of the Cκ and CH1 domains but have reduced pairing with CH1 and Cκ domains without the modifications. Such modifications can be particularly useful for preparing bispecific antibodies which two different pairs of Cκ and CH1 domains.

As demonstrated in the experimental examples, two groups of amino acids were identified as important interface residues which, when changed, can reduce or even disrupt the pairing of the Cκ and CH1 domains unless appropriate modifications are made to re-establish such interface.

One such group includes Val26 (Kabat numbering: Val133) and Phe11 (Kabat numbering: Phe118) of the Cκ domain and Leu11 (Kabat numbering: Leu124) of the CH1 domain. When one of these amino acids is substituted with Ala, for instance, the Cκ/CH1 pairing can be disrupted. Another example group includes Gln17 (Kabat numbering: 124) of Cκ and Phe9 (Kabat numbering: 122) of CH1.

Certain mutations at these interface residues, however, can restore the pairing, which is also demonstrated in the examples. One such example is Val26Trp (Cκ) with Leu11Trp (CH1). Further examples are shown in Table 1 and Table 2.

In one embodiment, provided is an antibody or antigen-binding fragment thereof, comprising a human CH1 fragment comprising a L11W substitution and a human Cκ fragment comprising a V26W substitution. Such an antibody or fragment can optionally include additional substitutions that further reduce the binding to the wild-type partner and/or enhance binding between the substituted fragments.

For instance, an additional pair of substitutions can be K101E in CH1 and D15K or D15H (D15K/H) in Cκ. Another pair of substitutions are K96D in CH1 and E16R in Cκ. Yet another example pair is K96E in CH1 and E16K in Cκ. Accordingly, in some embodiments, provided are antibody or antigen-binding fragment thereof, in which the CH1 fragment comprises substitutions L11W and K101E and the Cκ fragment comprises substitutions V26W and D15K/H; the CH1 fragment comprises substitutions L11W and K96D and the Cκ fragment comprises substitutions V26W and E16R; the CH1 fragment comprises substitutions L11W and K96E and the Cκ fragment comprises substitutions V26W and E16K; or the CH1 fragment comprises substitutions L11W and K96E and the Cκ fragment comprises substitutions V26W and E16R.

In one embodiment, provided is an antibody or antigen-binding fragment thereof, comprising a Cκ/CH1 pair, wherein the Cκ and CH1 fragments comprise amino acid residues selected from the group consisting of: (a) 26W in Cκ and 11K and 28N in CH1; (b) 11W and 26G in Cκ and 11W in CH1; (c) 26W in Cκ and 11W in CH1; (d) 17R in Cκ and 9D in CH1; (e) 17K in Cκ and 9D in CH1; and combinations thereof.

In some embodiments, the antibody or antigen-binding fragment thereof further comprises a second Cκ/CH1 pair. The second Cκ/CH1 pair can be wild-type or having a mutation group. The mutation group can be the same as in the first Cκ/CH1 pair but is preferable different such that there will not be mismatch between the pairs.

Another embodiment of the present disclosure provides an antibody or antigen-binding fragment thereof, comprising a Cκ domain comprising an amino acid modification at position V26 and/or F11, and a CH1 domain comprising an amino acid modification at position Leu11, wherein the modified amino acids interact with each other when the Cκ domain pairs with the CH1 domain. In some embodiments, the antibody or antigen-binding fragment thereof of claim 8, wherein the Cκ domain does not interact with a wild-type CH1 domain and the CH1 domain does not interact with a wild-type Cκ domain. In some embodiments, the modified amino acids are selected from Table 1.

Another embodiment provides an antibody or antigen-binding fragment thereof, comprising a Cκ domain comprising an amino acid modification at position Q17, and a CH1 domain comprising an amino acid modification at position F9, wherein the modified amino acids interact with each other when the Cκ domain pairs with the CH1 domain. In some embodiments, the Cκ domain does not interact with a wild-type CH1 domain and the CH1 domain does not interact with a wild-type Cκ domain. In some embodiments, the modified amino acids are selected from Table 2.

Also provided, in some embodiments, is a bispecific antibody comprising a first Cκ/CH1 pair and a second Cκ/CH1 pair, wherein the Cκ and CH1 fragments of the first pair comprise amino acid residues selected from the group consisting of: (a) 26W in Cκ and 11K and 28N in CH1; (b) 11W and 26G in Cκ and 11W in CH1; (c) 26W in Cκ and 11W in CH1; (d) 17R in Cκ and 9D in CH1; (e) 17K in Cκ and 9D in CH1; and combinations thereof, and the Cκ and CH1 fragments of the second pair are wild-type or comprise a different set of amino acid residues selected from (a)-(e).

### BRIEF DESCRIPTION OF THE DRAWINGS

FIG. 1 shows the crystal structure of a pair of Cκ and CH1 domains (from 1CZ8) showing their interactions (the residues involved in hydrogen bond are colored in pink; salt bridge in yellow; hydrophobic interaction residues are sticks colored in blue or green).

FIG. 2 shows a few residues in the Cκ and CH1 domain that may be important for maintaining the interaction between the domains

FIG. 3 presents the picture of a reduced SDS-PAGE gel for ala/trp mutations for different interaction amino acid pairs.

FIG. 4A-4D show the pictures of reduced SDS-PAGE gels for various mutation pair analyzed in Example 3.

FIG. 5A-B present pictures of reduced SDS-PAGE (5A) and non-reduced SDS-PAGE (5B) gels showing the binding between Cκ and CH1 domains.

FIG. 6A-C present gel images showing the binding between antibody heavy and light chains, some of which included mutations.

FIG. 7A-D illustrate the structures of a variety of bispecific antibodies.

FIG. 8A-B present data to show the binding and functional potency of the tested bispecific antibodies to their respective binding targets.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of“polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Biologically equivalent polynucleotides are those having the above-noted specified percent homology and encoding a polypeptide having the same or similar biological activity.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide often to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgGl, IgG2, IgG3, IgG4, IgAl and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See. e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

Some other numbering systems include “IMGT numbering” and “IMGT exon numbering. For example, for constant domains CH1 and Cκ, the following table shows the correlation between the IMGT exon numbering system and the Kabat numbering system.

IMGT exon numbering and Kabat numbering for CH1

| IMGT exon | Kabat |
|---|---|
| numbering | numbering |
| 1 | 114 |
| 2 | 115 |
| 3 | 116 |
| 4 | 117 |
| 5 | 118 |
| 6 | 119 |
| 7 | 120 |
| 8 | 121 |
| 9 | 122 |
| 10 | 123 |
| 11 | 124 |
| 12 | 125 |
| 13 | 126 |
| 14 | 127 |
| 15 | 128 |
| 16 | 129 |
| 17 | 130 |
| 18 | 133 |
| 19 | 134 |
| 20 | 135 |
| 21 | 136 |
| 22 | 137 |
| 23 | 138 |
| 24 | 139 |
| 25 | 140 |
| 26 | 141 |
| 27 | 142 |
| 28 | 143 |
| 29 | 144 |
| 30 | 145 |
| 31 | 146 |
| 32 | 147 |
| 33 | 148 |
| 34 | 149 |
| 35 | 150 |
| 36 | 151 |
| 37 | 152 |
| 38 | 153 |
| 39 | 154 |
| 40 | 156 |
| 41 | 157 |
| 42 | 162 |
| 43 | 163 |
| 44 | 164 |
| 45 | 165 |
| 46 | 166 |
| 47 | 167 |
| 48 | 168 |
| 49 | 169 |
| 50 | 171 |
| 51 | 172 |
| 52 | 173 |
| 53 | 174 |
| 54 | 175 |
| 55 | 176 |
| 56 | 177 |
| 57 | 178 |
| 58 | 179 |
| 59 | 180 |
| 60 | 182 |
| 61 | 183 |
| 62 | 184 |
| 63 | 185 |
| 64 | 186 |
| 65 | 187 |
| 66 | 188 |
| 67 | 189 |
| 68 | 190 |
| 69 | 191 |
| 70 | 192 |
| 71 | 193 |
| 72 | 194 |
| 73 | 195 |
| 74 | 196 |
| 75 | 197 |
| 76 | 198 |
| 77 | 199 |
| 78 | 200 |
| 79 | 203 |
| 80 | 205 |
| 81 | 206 |
| 82 | 207 |
| 83 | 208 |
| 84 | 209 |
| 85 | 210 |
| 86 | 211 |
| 87 | 212 |
| 88 | 213 |
| 89 | 214 |
| 90 | 215 |
| 91 | 216 |
| 92 | 217 |
| 93 | 218 |
| 94 | 219 |
| 95 | 220 |
| 96 | 221 |
| 97 | 222 |
| 98 | 223 |

IMGT exon numbering and Kabat numbering for Cκ

| IMGT exon | Kabat |
|---|---|
| numbering | numbering |
| 1 | 108 |
| 2 | 109 |
| 3 | 110 |
| 4 | 111 |
| 5 | 112 |
| 6 | 113 |
| 7 | 114 |
| 8 | 115 |
| 9 | 116 |
| 10 | 117 |
| 11 | 118 |
| 12 | 119 |
| 13 | 120 |
| 14 | 121 |
| 15 | 122 |
| 16 | 123 |
| 17 | 124 |
| 18 | 125 |
| 19 | 126 |
| 20 | 127 |
| 21 | 128 |
| 22 | 129 |
| 23 | 130 |
| 24 | 131 |
| 25 | 132 |
| 26 | 133 |
| 27 | 134 |
| 28 | 135 |
| 29 | 136 |
| 30 | 137 |
| 31 | 138 |
| 32 | 139 |
| 33 | 140 |
| 34 | 141 |
| 35 | 142 |
| 36 | 143 |
| 37 | 144 |
| 38 | 145 |
| 39 | 146 |
| 40 | 147 |
| 41 | 148 |
| 42 | 149 |
| 43 | 150 |
| 44 | 151 |
| 45 | 152 |
| 46 | 153 |
| 47 | 154 |
| 48 | 155 |
| 49 | 156 |
| 50 | 157 |
| 51 | 158 |
| 52 | 159 |
| 53 | 160 |
| 54 | 161 |
| 55 | 162 |
| 56 | 163 |
| 57 | 164 |
| 58 | 165 |
| 59 | 166 |
| 60 | 167 |
| 61 | 168 |
| 62 | 169 |
| 63 | 170 |
| 64 | 171 |
| 65 | 172 |
| 66 | 173 |
| 67 | 174 |
| 68 | 175 |
| 69 | 176 |
| 70 | 177 |
| 71 | 178 |
| 72 | 179 |
| 73 | 180 |
| 74 | 181 |
| 75 | 182 |
| 76 | 183 |
| 77 | 184 |
| 78 | 185 |
| 79 | 186 |
| 80 | 187 |
| 81 | 188 |
| 82 | 189 |
| 83 | 190 |
| 84 | 191 |
| 85 | 192 |
| 86 | 193 |
| 87 | 194 |
| 88 | 195 |
| 89 | 196 |
| 90 | 197 |
| 91 | 198 |
| 92 | 199 |
| 93 | 200 |
| 94 | 201 |
| 95 | 202 |
| 96 | 203 |
| 97 | 204 |
| 98 | 205 |
| 99 | 206 |
| 100 | 207 |
| 101 | 208 |
| 102 | 209 |
| 103 | 210 |
| 104 | 211 |
| 105 | 212 |
| 106 | 213 |
| 107 | 214 |

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG₁ molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CK regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

As used herein, “percent humanization” is calculated by determining the number of framework amino acid differences (i.e., non-CDR difference) between the humanized domain and the germline domain, subtracting that number from the total number of amino acids, and then dividing that by the total number of amino acids and multiplying by 100.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

Modified Cκ and CH1 domains

Bispecific antibodies (BsAbs), which target two antigens or epitopes, incorporate the specificities and properties of two distinct monoclonal antibodies (mAbs) into a single molecule. Mispairing may occur when there are two sets of paired VH-Ch1:VL-CL fragments. To avoid the mispairing of VH-CH1:VL-CL fragments derived from two distinct antibodies, a lot of methods have been used such as, Cross-Mab, common light chain, and FITIg.

An objective of the experimental examples was to introduce mutations into the Cκ and/or CH1 domain, in particular the human domains, to reduce mispairing. Preferably, the mutant Cκ can show good binding to the mutant CH1, but the mutant Cκ does not bind or has weak binding to the non-mutated CH1 domain and the mutant CH1 shows weak or no binding to the non-mutated Cκ.

First, important interface residues of human Cκ and CH1 were analyzed and five hotspots were discovered. To confirm the importance of these residues, mutations of each residue to alanine or tryptophan were prepared. Mutations at Gln17 of Cκ (Cκ_Q17) or Phe9 of CH1 (CH1_F9), and mutations at Val26 or Phe11 of Cκ (Cκ_V26_F11) or Leu11 of CH1 (CH1_L11) resulted in much decreased pairing of the light and heavy chains. These results confirmed that the groups Cκ_Q17/CH1_F9 (referred to as pair 1 in the examples) and Cκ_V26_F11/CH1_L11 (referred to as pair 2 in the examples) were important for the interaction of Cκ and CH1. Subsequently, mutations that could potentially restore the pairing were expressed and analyzed. Such modifications can be particularly useful for preparing bispecific antibodies which two different pairs of Cκ and CH1 domains.

For interface residues Cκ_V26_F11/CH1_L11 (and optionally L28), the following mutations are shown or contemplated to be able to restore the pairing of the Cκ and CH1 domains:

TABLE 1
Mutation Groups of Cκ at 26 and optionally at 11
with CH1 at 11 and optionally at 28

| No. | Cκ (at 26 and/or 11) | CH1 (at 11 and/or 28) |
|---|---|---|
| 1 | 26W | 11W |
| 2 | 26W | 11K_and 28N |
| 3 | 11W and 26G | 11W |
| 4 | 11W and 26G | 11K and 28N |
| 5 | 26F | 11F |
| 6 | 26W | 11F |
| 7 | 26F | 11W |
| 8 | 26L | 11W |
| 9 | 26M | 11W |
| 10 | 26E | 11W |
| 11 | 26W | 11W and 28R |
| 12 | 11A and 26W | 11W |

Likewise, for interface residues Cκ_Q17/CH1_F9, the following mutations are shown or contemplated to be able to restore the pairing of the Cκ and CH1 domains:

TABLE 2
Mutation Groups at Cκ 17/CH1 9

| No. | Cκ (at 17) | CH1 (at 9) |
|---|---|---|
| 1 | 17R | 9D |
| 2 | 17K | 9D |
| 3 | 17R | 9E |
| 4 | 17K | 9E |
| 5 | 17D | 9R |
| 6 | 17D | 9K |
| 7 | 17H | 9I |
| 8 | 17R | 9H |
| 9 | 17H | 9H |
| 10 | 17R | 9P |
| 11 | 17D | 9H |
| 12 | 17I | 9H |
| 13 | 17H | 9M |
| 14 | 17R | 9Q |
| 15 | 17H | 9Q |

As shown in Example 7, additional amino acid substitutions that disrupt one or more existing salt bridges in wild-type Cκ and CH1 domains and reestablish new ones can further improve the desired pairing specificity. The wild-type Cκ/CH1 pairs have salt bridges between CH1_K96 and Cκ_E16, between CH1_K101 and Cκ_D15, and between CH1_H51 and Cκ_D60. Each of these salt bridges can be suitable sites for substitutions.

For instance, in each of the salt bridges, the positively charged amino acid (e.g., K, R or H) can be substituted with a negatively charged amino acid (e.g., E or D), and the negatively amino acid (e.g., E or D) can be substituted with a positively charged amino acid (e.g., K, R, or H). One such example is CH1_K101E/Cκ_D15K or Cκ_D15H; another example is CH1_K96D/Cκ_E16R; another example is CH1_96E/Cκ_E16K; and another example is CH1_H51D/Cκ_D60K. These and other examples are illustrated in Table 3. Each of such substituted salt bridges can be used independently to prepare the new CH1/Cκ pairing, or in addition to any of the other substitutions described in the present disclosure.

TABLE 3
Disrupted and Reestablished Salt Bridges

| No. | CH1 | Cκ |
|---|---|---|
| 1 | K101E | D15H |
| 2 | K101E | D15K |
| 3 | K101E | D15R |
| 4 | K101D | D15H |
| 5 | K101D | D15K |
| 6 | K101D | D15R |
| 7 | K96D | E16R |
| 8 | K96E | E16K |
| 9 | K96D | E16K |
| 10 | K96E | E16R |
| 11 | K96D | E16H |
| 12 | K96E | E16H |
| 13 | H51D | D60K |
| 14 | H51D | D60R |
| 15 | H51D | D60H |
| 16 | H51E | D60K |
| 17 | H51E | D60R |
| 16 | H51E | D60H |

In one embodiment, a disclosed antibody or antigen-binding fragment thereof includes a CH1 fragment having substitutions L11W and K101E and a Cκ fragment having substitutions V26W and D15K/H. In one embodiment, a disclosed antibody or antigen-binding fragment thereof includes a CH1 fragment having substitutions L11W and K96D and a Cκ fragment having substitutions V26W and E16R. In one embodiment, a disclosed antibody or antigen-binding fragment thereof includes a CH1 fragment having substitutions L11W and K96E and a Cκ fragment having substitutions V26W and E16K.

These mutation groups can be useful for making mutated Cκ and CH1 domains that are able to bind each other, which cannot bind or have reduced binding to their wild type counterpart CH1 or Cκ domains. Such Cκ and CH1 domains can be incorporated into antibodies or antigen-binding fragments, in particular bispecific ones.

In one scenario, a bispecific antibody has a normal IgG structure which includes two light chain-heavy chain pairs. Each heavy chain includes a VH, CH1, CH2 and CH3 domains, and each light chain includes a VL and a CL (e.g., Cκ) domain. In accordance with one embodiment of the present disclosure, one of the Cκ/CH1 pairs includes a mutation group of the present disclosure and the other pair does not. In another embodiment, one of the Cκ/CH1 pairs includes a mutation group of the present disclosure and the other pair includes a different mutation group. In some embodiment, either of both of the pairs include two or more mutation groups (e.g., one group from Table 1 and another group from Table 2).

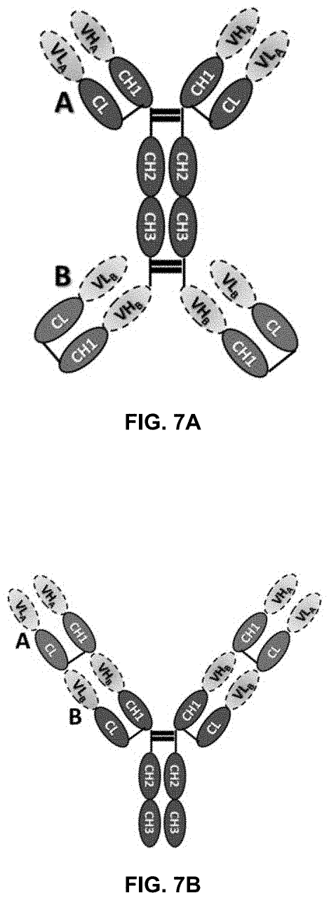

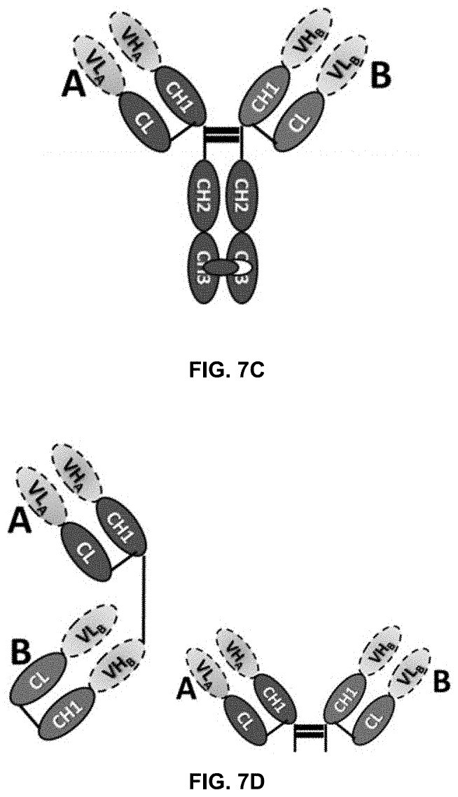

In another scenario, a bispecific antibody has a normal IgG structure which further is fused, at the C-terminus of the Fc fragment, to the N-termini of the VH's of a second Fab fragment. Such an antibody is illustrated in FIG. 7A. In accordance with one embodiment of the present disclosure, either of the Cκ/CH1 pairs at the N-terminal side of the Fc fragment or the Cκ/CH1 pairs at the C-terminal side of the Fc fragment includes a mutation group of the present disclosure and the other pairs do not. Furthermore, the mutation group can be included in both Cκ/CH1 pairs at the N or C-terminal side of the Fc fragment.

Yet in another embodiment, the bispecific antibody has a structure as illustrated in FIG. 7B. In this structure, each heavy chain and light chain includes two sets of concatenated Cκ/CH1 pairs. The mutation groups can be placed anywhere in this antibody so long as they favor the desired pairing. Another bispecific antibody, with a known knob-into-hole in the CH3 domains, is illustrated in FIG. 7C. Here, the mutation groups of the present disclosure can be inserted to either or both of the A and B Cκ/CH1 pairs. Yet other examples are illustrated in FIG. 7D which do not have CH2 or CH3 domains.

In one embodiment, the present disclosure provides an antibody or antigen-binding fragment thereof which includes a human Cκ/CH1 pair, wherein amino acid residue 26 of the Cκ domain is Trp and amino acid residue 11 of the CH1 domain is Trp. In some aspects, the antibody or antigen-binding fragment thereof further includes a second human Cκ/CH1 pair, wherein amino acid residue 26 of the second Cκ domain is not Trp and amino acid residue 11 of the second CH1 domain is not Trp. In some aspects, the antibody or antigen-binding fragment thereof further includes a heavy chain variable region, a light chain variable region, an Fc region, or the combination thereof.

In another embodiment, the present disclosure provides an antibody or antigen-binding fragment thereof, comprising a human Cκ domain comprising an amino acid modification at position Val26 and/or Phe11, and a human CH1 domain comprising an amino acid modification at position Leu11, wherein the modified amino acids interact with each other when the Cκ domain pairs with the CH1 domain. The amino modification, in some embodiments, is as compared to human IgG Cκ and CH1 domains. In some embodiments, the modified amino acids are selected from Table 1.

In some embodiments, the antibody or antigen-binding fragment thereof further includes a second Cκ/CH1 pair, wherein amino acid residue 26 of the second Cκ domain is Val and amino acid residue 11 of the second CH1 domain is Leu. In some aspects, amino acid residue 11 of the second Cκ domain is Phe.

In another embodiment, the present disclosure provides an antibody or antigen-binding fragment thereof, comprising a Cκ domain comprising an amino acid modification at position Gln17, and a CH1 domain comprising an amino acid modification at position Phe9, wherein the modified amino acids interact with each other when the Cκ domain pairs with the CH1 domain. The amino modification, in some embodiment, is as compared to human IgG Cκ and CH1 domains. In some embodiments, the modified amino acids are selected from Table 2.

In some embodiments, the antibody or antigen-binding fragment thereof further includes a second Cκ/CH1 pair, wherein amino acid residue 17 of the second Cκ domain is Gln and amino acid residue 9 of the second CH1 domain is Phe.

In some embodiments, the present disclosure provides an antibody or antigen-binding fragment thereof, which includes a mutation group of Table 1 or a mutation group of Table 2. In some embodiments, the antibody or antigen-binding fragment thereof includes a mutation group of Table 1 and a mutation group of Table 2. In some embodiments, the antibody or antigen-binding fragment thereof further includes a mutation group of Table 3.

the antibody or antigen-binding fragment thereof can be of any known class of antibodies, but is preferably of class IgG, including isotypes IgG1, IgG2, IgG3 and IgG4. The antibody or fragment thereof can be a chimeric antibody, a humanized antibody, or a fully human antibody.

### Bispecific/Bifunctional Molecules

Bispecific antibodies are provided in some embodiments. In some embodiments, the bispecific antibody has a first specificity to a tumor antigen or a microorganism. In some embodiments, the bispecific antibody has a second specificity to an immune cell.

In some embodiments, the immune cell is selected from the group consisting of a T cell, a B cell, a monocyte, a macrophage, a neutrophil, a dendritic cell, a phagocyte, a natural killer cell, an eosinophil, a basophil, and a mast cell. Molecules on the immune cell which can be targeted include, for example, CD3, CD16, CD19, CD28, and CD64. Other examples include PD-1, CTLA-4, LAG-3 (also known as CD223), CD28, CD122, 4-1BB (also known as CD137), TIM3, OX-40 or OX40L, CD40 or CD40L, LIGHT, ICOS/ICOSL, GITR/GITRL, TIGIT, CD27, VISTA, B7H3, B7H4, HEVM or BTLA (also known as CD272), killer-cell immunoglobulin-like receptors (KIRs), and CD47. Specific examples of bispecificity include, without limitation, PD-L1/PD-1, PD-L1/LAG3, PD-L1/TIGIT, and PD-L1/CD47.

A “tumor antigen” is an antigenic substance produced in tumor cells, i.e., it triggers an immune response in the host. Tumor antigens are useful in identifying tumor cells and are potential candidates for use in cancer therapy. Normal proteins in the body are not antigenic. Certain proteins, however, are produced or overexpressed during tumorigenesis and thus appear “foreign” to the body. This may include normal proteins that are well sequestered from the immune system, proteins that are normally produced in extremely small quantities, proteins that are normally produced only in certain stages of development, or proteins whose structure is modified due to mutation.

An abundance of tumor antigens are known in the art and new tumor antigens can be readily identified by screening. Non-limiting examples of tumor antigens include EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin.

Bifunctional molecules that include not just antibody or antigen binding fragment are also provided. As a tumor antigen targeting molecule, an antibody or antigen-binding fragment specific to PD-L1, such as those described here, can be combined with an immune cytokine or ligand optionally through a peptide linker. The linked immune cytokines or ligands include, but not limited to, IL-2, IL-3, IL-4, IL-5, IL-6, IL-7, IL-10, IL-12, IL-13, IL-15, GM-CSF, TNF-α, CD40L, OX40L, CD27L, CD30L, 4-1BBL, LIGHT and GITRL. Such bi-functional molecules can combine the immune checkpoint blocking effect with tumor site local immune modulation.

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

In certain embodiments, the prepared antibodies will not elicit a deleterious immune response in the animal to be treated, e.g., in a human. In one embodiment, antigen-binding polypeptides, variants, or derivatives thereof of the disclosure are modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. USA 57:6851-6855 (1984); Morrison et al., Adv. Immunol. 44:65-92 (1988); Verhoeyen et al., Science 239:1534-1536 (1988); Padlan, Molec. Immun. 25:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T-cell epitopes (see. e.g., International Application Publication Nos.: WO/9852976 A1 and WO/0034317 A2). For example, variable heavy chain and variable light chain sequences from the starting antibody are analyzed and a human T-cell epitope “map” from each V region showing the location of epitopes in relation to complementarity-determining regions (CDRs) and other key residues within the sequence is created. Individual T-cell epitopes from the T-cell epitope map are analyzed in order to identify alternative amino acid substitutions with a low risk of altering activity of the final antibody. A range of alternative variable heavy and variable light sequences are designed comprising combinations of amino acid substitutions and these sequences are subsequently incorporated into a range of binding polypeptides. Typically, between 12 and 24 variant antibodies are generated and tested for binding and/or function. Complete heavy and light chain genes comprising modified variable and human constant regions are then cloned into expression vectors and the subsequent plasmids introduced into cell lines for the production of whole antibody. The antibodies are then compared in appropriate biochemical and biological assays, and the optimal variant is identified.

The binding specificity of antigen-binding polypeptides of the present disclosure can be determined by in vitro assays such as immunoprecipitation, radioimmunoassay (RIA) or enzyme-linked immunoabsorbent assay (ELISA).

Alternatively, techniques described for the production of single-chain units (U.S. Pat. No. 4,694,778; Bird, Science 242:423-442 (1988); Huston et al., Proc. Natl. Acad. Sci. USA 55:5879-5883 (1988); and Ward et al., Nature 334:544-554 (1989)) can be adapted to produce single-chain units of the present disclosure. Single-chain units are formed by linking the heavy and light chain fragments of the Fv region via an amino acid bridge, resulting in a single-chain fusion peptide. Techniques for the assembly of functional Fv fragments in E. coli may also be used (Skerra et al., Science 242: 1038-1041 (1988)).

Examples of techniques which can be used to produce single-chain Fvs (scFvs) and antibodies include those described in U.S. Pat. Nos. 4,946,778 and 5,258,498; Huston et al., Methods in Enzymology 203:46-88 (1991); Shu et al., Proc. Natl. Sci. USA 90:1995-1999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See. e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816397, which are incorporated herein by reference in their entireties.

Humanized antibodies are antibody molecules derived from a non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen-binding. These framework substitutions are identified by methods well known in the art, e.g., by modeling of the interactions of the CDR and framework residues to identify framework residues important for antigen-binding and sequence comparison to identify unusual framework residues at particular positions. (See. e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Natl. Sci. USA 91:969-973 (1994)), and chain shuffling (U.S. Pat. No. 5,565,332, which is incorporated by reference in its entirety).

Completely human antibodies are particularly desirable for therapeutic treatment of human patients. Human antibodies can be made by a variety of methods known in the art including phage display methods using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see. e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can also be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 72:899-903 (1988). See also, U.S. Pat. No. 5,565,332, which is incorporated by reference in its entirety.)

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

Additionally, using routine recombinant DNA techniques, one or more of the CDRs of the antigen-binding polypeptides of the present disclosure, may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide, e.g., LIGHT. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present disclosure and within the skill of the art.

In addition, techniques developed for the production of “chimeric antibodies” (Morrison et al., Proc. Natl. Acad. Sci. USA: 851-855 (1984); Neuberger et al., Nature 372:604-608 (1984); Takeda et al., Nature 314:452-454 (1985)) by splicing genes from a mouse antibody molecule, of appropriate antigen specificity, together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

Alternatively, antibody-producing cell lines may be selected and cultured using techniques well known to the skilled artisan. Such techniques are described in a variety of laboratory manuals and primary publications. In this respect, techniques suitable for use in the disclosure as described below are described in Current Protocols in Immunology, Coligan et al., Eds., Green Publishing Associates and Wiley-Interscience, John Wiley and Sons, New York (1991) which is herein incorporated by reference in its entirety, including supplements.

Additionally, standard techniques known to those of skill in the art can be used to introduce mutations in the nucleotide sequence encoding an antibody of the present disclosure, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference variable heavy chain region, CDR-H1, CDR-H2, CDR-H3, variable light chain region, CDR-L1, CDR-L2, or CDR-L3. Alternatively, mutations can be introduced randomly along all or part of the coding sequence, such as by saturation mutagenesis, and the resultant mutants can be screened for biological activity to identify mutants that retain activity.

The present disclosure also provides pharmaceutical compositions. Such compositions comprise an effective amount of an antibody, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1: Cκ/CH1 Interface Interaction Analysis of Four Fab Fragments

This example analyzed a few antibody Fab fragments with respect to their Cκ/CH1 interface interactions.

### Structure 1: Interface Interaction Analysis for Cκ and CH1 of Fab 1F8

1F8 is a Fab molecule prepared from an antibody specific to human CD47. The complex crystal structure of the CD47 with anti-CD47 Fab 1F8 was conducted at a resolution of 3.1 A in 2017 (the light chain had 219 amino acids, where the Cκ included amino acids 114-219; the heavy chain had 220 amino acids, where the CH included amino acids 119-220).

In the interface between the Cκ and CH1 domains of this Fab fragment, there are a total of 32 residues from the CH domain and 35 residues from the Cκ domain. 1F8 has continuous residues between Ser14 and Gly20 in the CH domain. There is one more hydrogen bond formed between Lys16 main chain oxygen atom from the CH fragment and residue Lys100 from Cκ fragment, as compared to 4NYL (see structure 4 below). The hydrophobic interactions are similar to the other structures as shown below.

Hydrogen Bonds (distance cut-off: 3.5 Å)

| Cκ | | | CH1 | | | |
|---|---|---|---|---|---|---|
| | | Atom | | | Atom | Distance |
| Position | Residue | Name | Position | Residue | Name | (Å) |
| 16 | LYS | O | 100 | LYS | NZ | 3.4 |
| 30 | LYS | NZ | 24 | SER | OG | 2.7 |
| 51 | HIS | ND1 | 30 | ASN | OD1 | 3.3 |
| 54 | PRO | O | 55 | SER | OG | 2.7 |
| 57 | LEU | O | 53 | GLN | NE2 | 3.4 |
| 102 | SER | OG | 106 | GLU | O | 2.6 |

Notes:
1. HD between CH-Lys30 and Ser24 could be formed in the other three structures, as long as the NZ of Lys30 is rotated.
2. Extra HDs between CH-Lys16/CK-Lys100 and CH-Ser102/CK-Glu106 are formed because sequence difference than other 3 pdbs.

Salt Bridges between Cκ and CH1 of 1F8

| CH1 | | | Cκ | | | |
|---|---|---|---|---|---|---|
| | | Atom | | | Atom | Distance |
| Position | Residue | Name | Position | Residue | Name | (Å) |
| 96 | LYS | NZ | 16 | GLU | OE2 | 3.1 |
| 101 | LYS | NZ | 15 | ASP | OD2 | 3.8 |

Hydrophobic interface

| CH1 | | Cκ | | |
|---|---|---|---|---|
| Position | Residue | Position | Residue | Notes |
| 9 | PHE | 17 | GLN | Sandwich, more |
| | | | | like Van der Waals |
| 11 | LEU | 11, 26 | PHE, VAL | |
| 12 | ALA | 11 | PHE | |
| 24 | ALA | 9, 11 | PHE | |
| 53 | PHE | 28, 68, 69 | LEU, LEU, SER | |
| 68 | VAL | 28 | LEU | |

* Hydrophobic contacts involved in hydrogen bonds and salt bonds too are excluded in this table

Interfacing Residues in 1F8 CH1:

| | | | | | | Abs of |
|---|---|---|---|---|---|---|
| Position | Residue | Bond | ASA | BSA | DeltaG | DeltaG |
| 53 | PHE | | 104.91 | 102.42 | 1.64 | 1.64 |
| 9 | PHE | | 95.13 | 73.47 | 1.18 | 1.18 |
| 11 | LEU | | 63.14 | 60.63 | 0.97 | 0.97 |
| 56 | VAL | | 97.59 | 60.26 | 0.96 | 0.96 |
| 30 | LYS | H | 74.1 | 57.96 | −0.85 | 0.85 |
| 96 | LYS | S | 71.12 | 24.42 | −0.71 | 0.71 |
| 28 | LEU | | 48.35 | 42.65 | 0.68 | 0.68 |
| 24 | ALA | | 41.8 | 41.64 | 0.62 | 0.62 |
| 68 | VAL | | 41.46 | 36.31 | 0.58 | 0.58 |
| 54 | PRO | H | 118.8 | 51.46 | 0.53 | 0.53 |
| 16 | LYS | H | 190.06 | 97.08 | 0.44 | 0.44 |
| 19 | SER | | 87.49 | 29.98 | 0.37 | 0.37 |
| 10 | PRO | | 67.03 | 38.95 | 0.23 | 0.23 |
| 70 | THR | H | 74.01 | 32.39 | −0.16 | 0.16 |
| 57 | LEU | H | 101.62 | 7.97 | −0.09 | 0.09 |
| 51 | HIS | H | 125.59 | 86.42 | 0.08 | 0.08 |
| 58 | GLN | | 49.39 | 19.38 | 0.08 | 0.08 |
| 17 | SER | H | 44.25 | 44.25 | 0.06 | 0.06 |
| 18 | THR | H | 54.47 | 19.11 | −0.06 | 0.06 |
| 22 | THR | | 61.97 | 7.01 | −0.06 | 0.06 |
| 12 | ALA | | 72.88 | 29.29 | 0.05 | 0.05 |
| 66 | SER | | 30.01 | 25.56 | −0.05 | 0.05 |
| 52 | THR | | 60.18 | 4.28 | −0.04 | 0.04 |
| 59 | SER | | 129.9 | 3.35 | −0.04 | 0.04 |
| 25 | LEU | | 3.79 | 2.96 | 0.04 | 0.04 |
| 23 | ALA | | 2.05 | 1.88 | 0.03 | 0.03 |
| 8 | VAL | | 11.3 | 1.81 | −0.02 | 0.02 |
| 65 | LEU | | 13.88 | 1.01 | 0.02 | 0.02 |
| 14 | SER | | 52.86 | 6.52 | −0.01 | 0.01 |
| 15 | SER | | 98.56 | 0.61 | −0.01 | 0.01 |

Bond: bond type if formed hydrogen bond or salt bridge,
H: hydrogen bond,
S: salt bridge
ASA: accessible surface area
BSA: buried surface area
DeltaG: Change of Energy, positive involves more hydrophobic interaction while negative indicates more hydrophilic interaction
Abs of DeltaG: Absolute value of DeltaG, the table is sorted by this key. Residues (bold) with change of DeltaG above 0.5 can be regarded as the residues contribute more to stabilize the protein.

In the Cκ domain, seven residues are likely involved in interactions.

Interfacing residues in 1F8 Cκ:

| | | | | | | Abs of |
|---|---|---|---|---|---|---|
| Position | Residue | Bond | ASA | BSA | DeltaG | DeltaG |
| 11 | PHE | | 103.34 | 103.03 | 1.65 | 1.65 |
| 9 | PHE | | 85.86 | 83.98 | 1.34 | 1.34 |
| 100 | LYS | H | 86.5 | 41.41 | −1.06 | 1.06 |
| 57 | THR | | 76.88 | 61.43 | 0.93 | 0.93 |
| 28 | LEU | | 47.22 | 45.38 | 0.73 | 0.73 |
| 26 | VAL | | 42.67 | 42.67 | 0.68 | 0.68 |
| 53 | GLN | H | 153.38 | 81.8 | −0.65 | 0.65 |
| 14 | SER | | 63.01 | 48.31 | 0.47 | 0.47 |
| 30 | ASN | H | 46.04 | 36.92 | −0.44 | 0.44 |
| 102 | PHE | | 43.9 | 22.09 | 0.35 | 0.35 |
| 12 | PRO | | 78.85 | 40.42 | 0.31 | 0.31 |
| 16 | GLU | S | 132.97 | 48.56 | −0.31 | 0.31 |
| 101 | SER | | 64.98 | 27.71 | −0.31 | 0.31 |
| 31 | ASN | | 71.04 | 16.15 | −0.25 | 0.25 |
| 73 | THR | H | 78.11 | 22.7 | 0.18 | 0.18 |
| 17 | GLN | | 46.63 | 45.77 | 0.17 | 0.17 |
| 60 | ASP | | 67.72 | 10.4 | 0.14 | 0.14 |
| 67 | SER | | 21.05 | 20.24 | 0.13 | 0.13 |
| 69 | SER | | 30.67 | 27.47 | 0.13 | 0.13 |
| 7 | SER | | 56.42 | 8.61 | 0.12 | 0.12 |
| 54 | GLU | | 92.77 | 11.21 | −0.11 | 0.11 |
| 56 | VAL | | 43.54 | 12.76 | −0.1 | 0.1 |
| 71 | THR | | 39.29 | 14.82 | 0.09 | 0.09 |
| 10 | ILE | H | 28.88 | 27.62 | −0.07 | 0.07 |
| 58 | GLU | | 156.59 | 7.29 | 0.05 | 0.05 |
| 55 | SER | H | 73.31 | 57.53 | −0.04 | 0.04 |
| 24 | SER | H | 30.01 | 29.28 | 0.03 | 0.03 |
| 8 | VAL | | 9.41 | 1.5 | −0.02 | 0.02 |
| 68 | LEU | | 7.95 | 1.41 | 0.02 | 0.02 |
| 20 | SER | | 98.75 | 8.4 | −0.01 | 0.01 |
| 22 | THR | | 59.56 | 11.35 | −0.01 | 0.01 |
| 103 | ASN | | 47.6 | 0.87 | −0.01 | 0.01 |

Bond: bond type if formed hydrogen bond or salt bridge,
H: hydrogen bond,
S: salt bridge
ASA: accessible surface area
BSA: buried surface area
DeltaG: Change of Energy, positive involves more hydrophobic interaction while negative indicates more hydrophilic interaction
Abs of DeltaG: Absolute value of DeltaG, the table is sorted by this key. Residues (bold) with change of DeltaG above 0.5 can be regarded as the residues contribute more to stabilize the protein.

### Structure 2: Interface Interaction Analysis for Cκ and CH1 of 1CZ8

1CZ8 (PDB ID 1CZ8) is a Fab molecule prepared from an antibody specific to VEGF. The complex crystal structure of the VEGF and the Fab was conducted at a resolution of 2.4 A in year 2000.

Amino acid residues formed three antiparallel beta sheets in CH domain and four antiparallel beta sheets in the Cκ domain. These beta sheets formed a face-to-face conformation in the interface. In the interface between Cκ and CH1 domains of this Fab fragment, there are totally 28 residues from CH and 30 residues from CKdomain. There are three hydrogen bonds between the Cκ and CH1 domains. For example, in 1CZ8, CH residue His 51 and main chain oxygen atoms of Pro54 and Leu57 formed these three hydrogen bonds with Cκ residues Asn31, Ser55 and Gln53 respectively. These hydrogen binds are located on the one side of the interface.

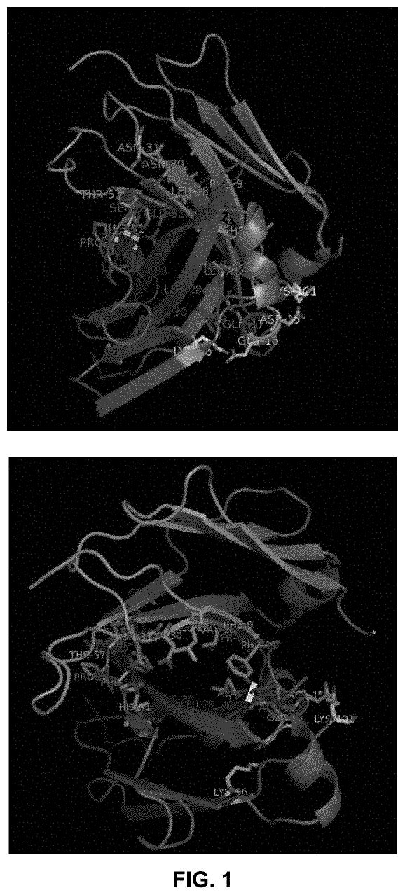

The hydrophobic interactions are mainly located at the central and other side of the interface, between CH residues Phe9, Leu11, Phe53, Val68 and Cκ residues Gln17, Phe11, Val26, Phe69 and Val28. Two salt bridges were formed between C-term of CH residues Lys96 and Lys101 and Cκ residue Asp15 and Glu16 to stabilize the CH and Cκ complex structure on the other side of the interface (FIG. 1; residues involved in hydrogen bond colored in pink; salt bridge in yellow; hydrophobic interaction residues are sticks colored in blue or green).

Hydrogen Bonds (distance cut-off: 3.5 Å)

| CH1 | | | Cκ | | | |
|---|---|---|---|---|---|---|
| | | Atom | | | Atom | Distance |
| Position | Residue | Name | Position | Residue | Name | (Å) |
| 51 | HIS | NE2 | 31 | ASN | OD1 | 2.86 |
| 54 | PRO | O | 55 | SER | OG | 2.6 |
| 57 | LEU | O | 53 | GLN | NE2 | 2.9 |

Water-mediated hydrogen binding

| | | | | | |
|---|---|---|---|---|---|
| 10 | PRO | O | 12 | PRO | O |
| 58 | GLN | OE1 | 24 | SER | OG |
| 54 | PRO | O | 71 | THR | OG1 |

Salt Bridges between CH and Cκ

| CH1 | | | Cκ | | | |
|---|---|---|---|---|---|---|
| | | Atom | | | Atom | Distance |
| Position | Residue | Name | Position | Residue | Name | (Å) |
| 96 | LYS | NZ | 16 | GLU* | OE1 | 3.0 |
| 101 | LYS | NZ | 15 | ASP* | OD1 | 2.8 |

Hydrophobic interface (distance cut-off: 4 Å)

| CH1 | | Cκ | | |
|---|---|---|---|---|
| Position | Residue | Position | Residue | Notes |
| 9 | PHE | 17 | Gln | |
| 11 | LEU | 11, 26 | PHE, VAL | 4A from Leu15 Ca atom |
| 12 | ALA | 11 | PHE | |
| 24 | ALA | 11 | PHE | Displaced |
| 53 | PHE | 28, 69 | LEU, SER | Sandwich |
| 68 | VAL | 28 | LEU | Sandwich |

Top 5 important interface residues for Cκ and CH1 interaction

| CH1 | | Cκ | |
|---|---|---|---|
| Position | Residue | Position | Residue |
| 51 | HIS | 31 | ASN |
| 57 | LEU | 53 | GLN |
| 9 | PHE | 17 | GLN |
| 53 | PHE | 69 | SER |
| 11 | LEU | 11, 26 | PHE, VAL |

Note:
Salt bridge residues are excluded

Free energy deviation analysis identified some residues in 1cz8 CH1 have stronger interactions with Cκ residues (see the first 9 residues in the table below, bolded).

Interfacing Residues: 1cz8 CH1

| Position | Residue | bond | ASA | BSA | DeltaG | Abs of DeltaG |
|---|---|---|---|---|---|---|
| 53 | PHE | | 103.02 | 99.71 | 1.6 | 1.6 |
| 9 | PHE | | 96.3 | 77.27 | 1.24 | 1.24 |
| 11 | LEU | | 64.71 | 61.37 | 0.98 | 0.98 |
| 56 | VAL | | 93.45 | 56.39 | 0.9 | 0.9 |
| 28 | LEU | | 48.79 | 44.61 | 0.71 | 0.71 |
| 51 | HIS | H | 114.24 | 93.31 | 0.68 | 0.68 |
| 54 | PRO | H | 120.4 | 53.11 | 0.59 | 0.59 |
| 68 | VAL | | 35.98 | 34.81 | 0.56 | 0.56 |
| 24 | ALA | | 53.89 | 51.35 | 0.55 | 0.55 |
| 70 | THR | | 64.58 | 33.59 | 0.43 | 0.43 |
| 96 | LYS | S | 63.68 | 16.19 | −0.39 | 0.39 |
| 101 | LYS | S | 231.91 | 46.46 | −0.39 | 0.39 |
| 30 | LYS | | 62.59 | 47.59 | 0.29 | 0.29 |
| 10 | PRO | | 66.28 | 37.49 | 0.26 | 0.26 |
| 12 | ALA | | 47.32 | 20.55 | −0.2 | 0.2 |
| 57 | LEU | H | 101.85 | 12.03 | −0.14 | 0.14 |
| 66 | SER | | 28.08 | 23.48 | 0.13 | 0.13 |
| 58 | GLN | | 41.61 | 13.28 | 0.11 | 0.11 |
| 8 | VAL | | 16.24 | 6.24 | −0.07 | 0.07 |
| 25 | LEU | | 3.82 | 3.49 | 0.06 | 0.06 |
| 23 | ALA | | 14.88 | 3.31 | 0.05 | 0.05 |
| 59 | SER | | 132.12 | 3.5 | −0.04 | 0.04 |
| 52 | THR | | 59.64 | 4.29 | −0.03 | 0.03 |
| 22 | THR | | 97.5 | 16.73 | 0.02 | 0.02 |
| 64 | SER | | 11.46 | 1.84 | −0.02 | 0.02 |
| 13 | PRO | | 5.85 | 0.5 | 0.01 | 0.01 |
| 14 | SER | | 149.42 | 0.94 | 0.01 | 0.01 |

Bond: bond type if formed hydrogen bond or salt bridge, H: hydrogen bond, S: salt bridge
ASA: accessible surface area
BSA: buried surface area
DeltaG: Change of Energy, positive involves more hydrophobic interaction while negative indicates more hydrophilic interaction
Abs of DeltaG: Absolute value of DeltaG, the table is sorted by this key. Residues (bold) with change of DeltaG above 0.5 can be regarded as the residues contribute more to stabilize the protein.

In the Cκ domain, five residues are likely involved in interactions.

Interfacing Residues: 1cz8 Cκ

| Position | Residue | bond | ASA | BSA | DeltaG | Abs of DeltaG |
|---|---|---|---|---|---|---|
| 11 | PHE | | 100.45 | 95.29 | 1.52 | 1.52 |
| 9 | PHE | | 99.99 | 59.32 | 0.95 | 0.95 |
| 28 | LEU | | 48.88 | 48.38 | 0.77 | 0.77 |
| 26 | VAL | | 46.36 | 45.71 | 0.73 | 0.73 |
| 53 | GLN | H | 152.03 | 80.54 | −0.63 | 0.63 |
| 14 | SER | | 62.19 | 48.96 | 0.49 | 0.49 |
| 30 | ASN | | 45.99 | 39.43 | −0.49 | 0.49 |
| 16 | GLU | S | 133.88 | 61.31 | −0.46 | 0.46 |
| 15 | ASP | S | 118.39 | 37.99 | −0.36 | 0.36 |
| 69 | SER | | 37.96 | 30.45 | 0.3 | 0.3 |
| 31 | ASN | H | 71 | 17.38 | −0.27 | 0.27 |
| 67 | SER | | 20.55 | 20.55 | 0.18 | 0.18 |
| 17 | GLN | | 55.44 | 53.37 | 0.16 | 0.16 |
| 56 | VAL | | 64.32 | 17.9 | −0.14 | 0.14 |
| 71 | THR | | 44.12 | 16.45 | 0.12 | 0.12 |
| 60 | ASP | | 61.58 | 15.19 | −0.11 | 0.11 |
| 54 | GLU | | 92.25 | 12.17 | −0.1 | 0.1 |
| 22 | THR | | 65.41 | 8.27 | 0.07 | 0.07 |
| 57 | THR | | 59.04 | 44.28 | −0.07 | 0.07 |
| 68 | LEU | | 7.25 | 2.72 | 0.04 | 0.04 |
| 20 | SER | | 84.08 | 5.75 | −0.02 | 0.02 |
| 58 | GLU | | 146.27 | 4.53 | 0.02 | 0.02 |
| 10 | ILE | | 25.87 | 0.86 | −0.01 | 0.01 |
| 13 | PRO | | 12.65 | 1.66 | −0.01 | 0.01 |
| 24 | SER | | 31.09 | 30.1 | 0.01 | 0.01 |
| 55 | SER | H | 71.35 | 56.44 | −0.01 | 0.01 |
| 73 | THR | | 81.21 | 12.51 | −0.01 | 0.01 |

Bond: bond type if formed hydrogen bond or salt bridge, H: hydrogen bond, S: salt bridge
ASA: accessible surface area
BSA: buried surface area
DeltaG: Change of Energy, positive involves more hydrophobic interaction while negative indicates more hydrophilic interaction
Abs of DeltaG: Absolute value of DeltaG, the table is sorted by this key. Residues (bold) with change of DeltaG above 0.5 can be regarded as the residues contribute more to stabilize the protein.

### Structure 3: Interface Interaction Analysis for Cκ and CH1 of 1L7I

1L7I is a known Fab molecule (PDB ID: 1L7I) targeting ErbB2. The crystal Structure of this anti-ErbB2 Fab2C4 was resolved at 1.8 A in year 2002.

In the interface between Cκ and CH1 domain of this Fab fragment (PDB ID 1L7i), there are total 33 residues from CH and 35 residues from Cκ domain.

Hydrogen Bonds of 1L7i (distance cut-off: 3.5 Å)

| Cκ | | | CH1 | | | |
|---|---|---|---|---|---|---|
| | | Atom | | | Atom | Distance |
| Position | Residue | Name | Position | Residue | Name | (Å) |
| 16 | LYS | O | 10 | ILE | N | 3.16 |
| 16 | LYS | NZ | 101 | SER | O | 2.99 |
| 51 | HIS | ND1 | 31 | ASN | OD1 | 3.2 |
| 54 | PRO | O | 55 | SER | OG | 2.7 |
| 57 | LEU | O | 53 | GLN | NE2 | 2.9 |

Water-mediated hydrogen binding

| | | | | | |
|---|---|---|---|---|---|
| 10 | PRO | O | 12 | PRO | O |
| 12 | ALA | O | 10 | ILE | O |
| 54 | PRO | O | 71 | THR | OG1 |

Salt Bridges between CK and CH of 1L7i

| CH1 | | | Cκ | | | |
|---|---|---|---|---|---|---|
| | | Atom | | | Atom | Distance |
| Position | Residue | Name | Position | Residue | Name | (Å) |
| 96 | LYS | NZ | 16 | GLU | OE1, OE2 | 3.4-2.7 |

Note:
C-term residues Cys 103 of CH and Cys 107 CK formed a disulfide bridge which broke the salt bridge between CH residue Lys101 and Ck residue Asp15 which was seen in other structures.

Hydrophobic interface of 1L7i

| Cκ | | | CH1 | | | |
|---|---|---|---|---|---|---|
| | | Atom | | | Atom | Distance |
| Position | Residue | Name | Position | Residue | Name | (Å) |
| 16 | LYS | O | 100 | LYS | NZ | 3.4 |
| 30 | LYS | NZ | 24 | SER | OG | 2.7 |
| 51 | HIS | ND1 | 30 | ASN | OD1 | 3.3 |
| 54 | PRO | O | 55 | SER | OG | 2.7 |
| 57 | LEU | O | 53 | GLN | NE2 | 3.4 |
| 102 | SER | OG | 106 | GLU | O | 2.6 |

Free energy deviation analysis identified some residues in 1L7i CH1 have stronger interactions with Cκ residues (see the first 12 residues in the table below, bolded).

Interfacing Residues: 1L7i CH1

| Position | Residue | bond | ASA | BSA | DeltaG | Abs of DeltaG |
|---|---|---|---|---|---|---|
| 103 | CYS | | 113.02 | 79.88 | 2.31 | 2.31 |
| 53 | PHE | | 104.25 | 101.74 | 1.63 | 1.63 |
| 9 | PHE | | 99.04 | 80.13 | 1.28 | 1.28 |
| 101 | LYS | S | 141.67 | 60.98 | −1.2 | 1.2 |
| 56 | VAL | | 92.31 | 59.34 | 0.95 | 0.95 |
| 11 | LEU | | 61.26 | 57.09 | 0.91 | 0.91 |
| 54 | PRO | H | 116.04 | 53.67 | 0.73 | 0.73 |
| 28 | LEU | | 50.04 | 45.19 | 0.72 | 0.72 |
| 17 | SER | | 37.12 | 37.12 | 0.55 | 0.55 |
| 68 | VAL | | 34.63 | 33.97 | 0.54 | 0.54 |
| 24 | ALA | | 33.93 | 33.93 | 0.52 | 0.52 |
| 96 | LYS | S | 69.62 | 16.57 | −0.52 | 0.52 |
| 70 | THR | | 59.2 | 34.05 | 0.42 | 0.42 |
| 10 | PRO | | 57.58 | 41.55 | 0.33 | 0.33 |
| 30 | LYS | | 64.74 | 49.18 | 0.29 | 0.29 |
| 58 | GLN | | 48.94 | 23.52 | 0.26 | 0.26 |
| 16 | LYS | H | 154.48 | 98.09 | 0.21 | 0.21 |
| 12 | ALA | | 37.27 | 23.91 | −0.17 | 0.17 |
| 66 | SER | | 26.68 | 23.26 | 0.14 | 0.14 |
| 22 | THR | | 61.32 | 9.4 | 0.13 | 0.13 |
| 102 | SER | | 106.39 | 12.64 | −0.12 | 0.12 |
| 57 | LEU | | 109.6 | 10.1 | −0.11 | 0.11 |
| 18 | THR | | 47.02 | 7.72 | −0.09 | 0.09 |
| 19 | SER | | 89.59 | 40.33 | −0.07 | 0.07 |
| 25 | LEU | | 4.95 | 4.61 | 0.07 | 0.07 |
| 15 | SER | | 83.74 | 3.93 | −0.04 | 0.04 |
| 14 | SER | | 15.02 | 4.4 | −0.03 | 0.03 |
| 51 | HIS | | 111.16 | 79.43 | 0.02 | 0.02 |
| 52 | THR | | 62.51 | 3.83 | −0.02 | 0.02 |
| 23 | ALA | | 0.33 | 0.33 | 0.01 | 0.01 |
| 59 | SER | | 127.52 | 1.31 | −0.01 | 0.01 |
| 64 | SER | | 9.13 | 0.61 | −0.01 | 0.01 |

Bond: bond type if formed hydrogen bond or salt bridge, H: hydrogen bond, S: salt bridge
ASA: accessible surface area
BSA: buried surface area
DeltaG: Change of Energy, positive involves more hydrophobic interaction while negative indicates more hydrophilic interaction
Abs of DeltaG: Absolute value of DeltaG, the table is sorted by this key. Residues (bold) with change of DeltaG above 0.5 can be regarded as the residues contribute more to stabilize the protein.

In the Cκ domain, nine residues are likely involved in interactions.

Interfacing Residues: 1L7i Cκ

| Position | Residue | bond | ASA | BSA | DeltaG | Abs of DeltaG |
|---|---|---|---|---|---|---|
| 11 | PHE | | 105.85 | 105.73 | 1.68 | 1.68 |
| 9 | PHE | | 89.04 | 88.03 | 1.41 | 1.41 |
| 100 | LYS | H | 85.84 | 42.05 | −1.08 | 1.08 |
| 57 | THR | | 74.5 | 58.15 | 0.89 | 0.89 |
| 107 | CYS | S | 101.84 | 64.95 | 0.89 | 0.89 |
| 28 | LEU | | 48.03 | 47.86 | 0.77 | 0.77 |
| 26 | VAL | | 44.53 | 44.19 | 0.71 | 0.71 |
| 53 | GLN | | 151.85 | 79.95 | −0.58 | 0.58 |
| 12 | PRO | | 56.22 | 48.12 | 0.54 | 0.54 |
| 30 | ASN | | 43.79 | 36.72 | −0.44 | 0.44 |
| 16 | GLU | S | 108.76 | 56.23 | −0.4 | 0.4 |
| 14 | SER | | 53.87 | 41.73 | 0.39 | 0.39 |
| 69 | SER | | 41 | 33.43 | 0.33 | 0.33 |
| 31 | ASN | | 78.35 | 17.4 | −0.27 | 0.27 |
| 101 | SER | | 57.64 | 22.68 | −0.26 | 0.26 |
| 102 | PHE | | 21.99 | 16.51 | 0.26 | 0.26 |
| 73 | THR | | 63.23 | 17.38 | 0.15 | 0.15 |
| 60 | ASP | | 61.68 | 11.2 | −0.14 | 0.14 |
| 7 | SER | | 48.66 | 8.03 | 0.13 | 0.13 |
| 67 | SER | | 16.14 | 16.14 | 0.13 | 0.13 |
| 56 | VAL | | 37.56 | 12.43 | −0.12 | 0.12 |
| 106 | GLU | | 136 | 18.91 | −0.12 | 0.12 |
| 55 | SER | H | 69.61 | 59.4 | 0.11 | 0.11 |
| 13 | PRO | | 14.76 | 6.87 | −0.08 | 0.08 |
| 17 | GLN | | 49.85 | 48.28 | 0.08 | 0.08 |
| 24 | SER | | 24.37 | 22.04 | 0.08 | 0.08 |
| 54 | GLU | | 96.84 | 10.59 | −0.07 | 0.07 |
| 8 | VAL | | 15.41 | 5.63 | −0.06 | 0.06 |
| 71 | THR | | 41.6 | 14.33 | 0.06 | 0.06 |
| 58 | GLU | | 156.39 | 9.76 | 0.04 | 0.04 |
| 10 | ILE | H | 31.71 | 31.59 | 0.03 | 0.03 |
| 15 | ASP | | 107.21 | 9.87 | 0.03 | 0.03 |
| 68 | LEU | | 4.61 | 1.93 | 0.03 | 0.03 |
| 22 | THR | | 52.89 | 8.19 | 0.02 | 0.02 |
| 20 | SER | | 84.54 | 13.16 | 0.01 | 0.01 |

Bond: bond type if formed hydrogen bond or salt bridge, H: hydrogen bond, S: salt bridge
ASA: accessible surface area
BSA: buried surface area
DeltaG: Change of Energy, positive involves more hydrophobic interaction while negative indicates more hydrophilic interaction
Abs of DeltaG: Absolute value of DeltaG, the table is sorted by this key. Residues (bold) with change of DeltaG above 0.5 can be regarded as the residues contribute more to stabilize the protein.

### Structure 4: Interface Interaction Analysis for Cκ and CH1 of 4NYL

The fourth structure being studied was 4NYL, a known Fab molecule (PDB ID: 4NYL), targeting TNFa. The crystal structure of the adalimumab FAB fragment was resolved at 2.8 A in year 2014 (solved with a relative high Rfree (Rfree=35.8%/R=27.5), which means that the structure is not suitable for detailed analysis). Adalimumab is antibody against TNFa, used to treat patients with rheumatoid arthritis, psoriatic arthritis and ankylosing spondylitis, and children with juvenile idiopathic arthritis. In the interface between Cκ and CH1 domain of adalimumab Fab fragment (PDB ID 4NYL), there are total 24 residues from CH1 and 28 residues from Cκ domain.

4NYL has the same hydrogen bond and hydrophobic interaction as that in 1CZ8. Due to the lack of C-term Ch residues, only one salt bridge was formed between C-term of CH residue Lys96 and Cκ residue Glu15.

Hydrogen Bonds of 4NYL (distance cut-off: 3.5 Å)

| CH1 | | | Cκ + Vκ | | | |
|---|---|---|---|---|---|---|
| | | Atom | | | Atom | Distance |
| Position | Residue | Name | Position | Residue | Name | (Å) |
| 51 | HIS | NE2 | 30 | ASN | OD1 | 3.22 |
| 54 | PRO | O | 55 | SER | OG | 2.7 |
| 57 | LEU | O | 53 | GLN | OE1 | 2.97 |

Note:
due to resolution limit, no water mediated hydrogen bonds are found.

Salt Bridges between CK and CH of 4NYL

| CH1 | | | Cκ | | | |
|---|---|---|---|---|---|---|
| | | Atom | | | Atom | Distance |
| Position | Residue | Name | Position | Residue | Name | (Å) |
| 96 | LYS | NZ | 16 | GLU | OE1, OE2 | 3.4-2.7 |

Note:
As 4NYL has C-term residues 100-103 missing, so salt bridge between CH-Lys101 and CK-Asp15 is missing.

Hydrophobic interface of 4NYL

| CH1 | | Cκ | | |
|---|---|---|---|---|
| Position | Residue | Position | Residue | Notes |
| 9 | PHE | 17 | GLN | Sandwich |
| 11 | LEU | 11, 26 | PHE, VAL | Sandwich |
| 12 | ALA | 11 | PHE | displace |
| 24 | ALA | 9, 11, 28 | PHE, LEU, LEU | Sandwich |
| 68 | VAL | 28 | LEU | Sandwich |

Free energy deviation analysis identified some residues in 4NYL CH1 have stronger interactions with Cκ residues (see the first nine residues in the table below, bolded).

Interfacing Residues: 4NYL CH1

| Position | Residue | bond | ASA | BSA | DeltaG | Abs of DeltaG |
|---|---|---|---|---|---|---|
| 53 | PHE | | 96.83 | 95.9 | 1.53 | 1.53 |
| 9 | PHE | | 97.57 | 74.98 | 1.2 | 1.2 |
| 11 | LEU | | 67.89 | 65.22 | 1.04 | 1.04 |
| 56 | VAL | | 102.16 | 64.24 | 1.03 | 1.03 |
| 28 | LEU | | 56.92 | 51.23 | 0.82 | 0.82 |
| 54 | PRO | H | 117.72 | 48.38 | 0.65 | 0.65 |
| 68 | VAL | | 38.86 | 38.35 | 0.61 | 0.61 |
| 24 | ALA | | 56.65 | 54.38 | 0.59 | 0.59 |
| 51 | HIS | H | 109.04 | 76.51 | 0.54 | 0.54 |
| 70 | THR | | 65.96 | 30.78 | 0.47 | 0.47 |
| 12 | ALA | | 65.59 | 34.43 | −0.27 | 0.27 |
| 10 | PRO | | 58.21 | 35.55 | 0.21 | 0.21 |
| 96 | LYS | | 71.02 | 8.03 | 0.13 | 0.13 |
| 13 | PRO | | 110.53 | 7.16 | 0.11 | 0.11 |
| 58 | GLN | | 45.51 | 21.8 | 0.11 | 0.11 |
| 52 | THR | | 68.63 | 7.32 | −0.08 | 0.08 |
| 57 | LEU | H | 105.07 | 6.99 | −0.08 | 0.08 |
| 25 | LEU | | 10.31 | 4.61 | 0.07 | 0.07 |
| 66 | SER | | 27.04 | 21.25 | 0.07 | 0.07 |
| 23 | ALA | | 20.19 | 3.62 | 0.06 | 0.06 |
| 22 | THR | | 99.8 | 17.73 | 0.04 | 0.04 |
| 59 | SER | | 129.22 | 4.43 | −0.04 | 0.04 |
| 30 | LYS | | 68.02 | 48.93 | −0.03 | 0.03 |
| 64 | SER | | 15.79 | 1.32 | −0.01 | 0.01 |

Bond: bond type if formed hydrogen bond or salt bridge, H: hydrogen bond, S: salt bridge
ASA: accessible surface area
BSA: buried surface area
DeltaG: Change of Energy, positive involves more hydrophobic interaction while negative indicates more hydrophilic interaction
Abs of DeltaG: Absolute value of DeltaG, the table is sorted by this key. Residues (bold) with change of DeltaG above 0.5 can be regarded as the residues contribute more to stabilize the protein.

In the Cκ domain, seven residues are likely involved in interactions.

Interfacing Residues: 4NYL Cκ

| Position | Residue | bond | ASA | BSA | DeltaG | Abs of DeltaG |
|---|---|---|---|---|---|---|
| 11 | PHE | | 94.4 | 92.85 | 1.49 | 1.49 |
| 9 | PHE | | 98.42 | 57.04 | 0.91 | 0.91 |
| 28 | LEU | | 53.03 | 53.03 | 0.85 | 0.85 |
| 57 | THR | | 77.07 | 52.33 | 0.81 | 0.81 |
| 26 | VAL | | 46.03 | 45.87 | 0.73 | 0.73 |
| 53 | GLN | H | 146.87 | 74.61 | −0.59 | 0.59 |
| 30 | ASN | | 53.19 | 42.51 | −0.51 | 0.51 |
| 14 | SER | | 73.33 | 53.83 | 0.41 | 0.41 |
| 16 | GLU | | 81.87 | 23.61 | 0.36 | 0.36 |
| 69 | SER | | 36.91 | 32.06 | 0.36 | 0.36 |
| 31 | ASN | H | 68.16 | 17.44 | −0.21 | 0.21 |
| 22 | THR | | 53.37 | 11.88 | 0.19 | 0.19 |
| 67 | SER | | 17.29 | 16.83 | 0.15 | 0.15 |
| 20 | SER | | 77.72 | 8.53 | 0.14 | 0.14 |
| 12 | PRO | | 72.19 | 25.45 | 0.12 | 0.12 |
| 56 | VAL | | 66.37 | 16.66 | −0.12 | 0.12 |
| 60 | ASP | | 61.84 | 6.5 | −0.11 | 0.11 |
| 73 | THR | | 69.46 | 16.93 | 0.1 | 0.1 |
| 17 | GLN | | 47.27 | 41.79 | 0.08 | 0.08 |
| 24 | SER | | 37.71 | 35.01 | 0.05 | 0.05 |
| 54 | GLU | | 93.02 | 10.6 | −0.05 | 0.05 |
| 58 | GLU | | 154.24 | 3.58 | 0.05 | 0.05 |
| 10 | ILE | | 33.1 | 3.56 | −0.04 | 0.04 |
| 55 | SER | H | 70.17 | 60.32 | 0.04 | 0.04 |
| 13 | PRO | | 16.67 | 1.84 | 0.03 | 0.03 |
| 68 | LEU | | 7.35 | 1.92 | 0.03 | 0.03 |
| 71 | THR | | 45.38 | 15.86 | 0.01 | 0.01 |

Bond: bond type if formed hydrogen bond or salt bridge, H: hydrogen bond S: salt bridge
ASA: accessible surface area
BSA: buried surface area
DeltaG: Change of Energy, positive involves more hydrophobic interaction while negative indicates more hydrophilic interaction
Abs of DeltaG: Absolute value of DeltaG, the table is sorted by this key. Residues (bold) with change of DeltaG above 0.5 can be regarded as the residues contribute more to stabilize the protein.

Interface Analysis for CH1_Cκ of 1cz8, 4nyl, 1l7i, hCD47-1_1F8

Interface analysis for the above four structures includes salt bridge, hydrogen bond and hydrophobic interaction. All of the DeltaG were calculated and the amino acids were ranked by DeltaG. For each structure, Top10 pairs were chosen for further analysis. The analysis focused on hydrophobic interaction regardless of other interactions. Then Top5 pairs were selected for lead candidates.

Sequence recoding of CH1

| Recoding | | 1cz8 | 1l7i | 4nyl | 1F8 |
|---|---|---|---|---|---|
| 1 | ALA | ALA 124 | ALA 114 | ALA 122 | ALA 119 |
| 2 | SER | SER 125 | SER 115 | SER 123 | SER 120 |
| 3 | THR | THR 126 | THR 116 | THR 124 | THR 121 |
| 4 | LYS | LYS 127 | LYS 117 | LYS 125 | LYS 122 |
| 5 | GLY | GLY 128 | GLY 118 | GLY 126 | GLY 123 |
| 6 | PRO | PRO 129 | PRO 119 | PRO 127 | PRO 124 |
| 7 | SER | SER 130 | SER 120 | SER 128 | SER 125 |
| 8 | VAL | VAL 131 | VAL 121 | VAL 129 | VAL 126 |
| 9 | PHE | PHE 132 | PHE 122 | PHE 130 | PHE 127 |
| 10 | PRO | PRO 133 | PRO 123 | PRO 131 | PRO 128 |
| 11 | LEU | LEU 134 | LEU 124 | LEU 132 | LEU 129 |
| 12 | ALA | ALA 135 | ALA 125 | ALA 133 | ALA 130 |
| 13 | PRO | PRO 136 | PRO 126 | PRO 134 | PRO 131 |
| 14 | SER | SER 137 | SER 127 | / | SER 132 |
| 15 | SER | / | SER 128 | / | SER 133 |
| 16 | LYS | / | LYS 129 | / | LYS 134 |
| 17 | SER | / | SER 130 | / | SER 135 |
| 18 | THR | / | THR 131 | / | THR 136 |
| 19 | SER | / | SER 132 | / | SER 137 |
| 20 | GLY | / | GLY 133 | / | GLY 138 |
| 21 | GLY | GLY 144 | GLY 134 | GLY 142 | GLY 139 |
| 22 | THR | THR 145 | THR 135 | THR 143 | THR 140 |
| 23 | ALA | ALA 146 | ALA 136 | ALA 144 | ALA 141 |
| 24 | ALA | ALA 147 | ALA 137 | ALA 145 | ALA 142 |
| 25 | LEU | LEU 148 | LEU 138 | LEU 146 | LEU 143 |
| 26 | GLY | GLY 149 | GLY 139 | GLY 147 | GLY 144 |
| 27 | CYS | CYS 150 | CYS 140 | CYS 148 | CYS 145 |
| 28 | LEU | LEU 151 | LEU 141 | LEU 149 | LEU 146 |
| 29 | VAL | VAL 152 | VAL 142 | VAL 150 | VAL 147 |
| 30 | LYS | LYS 153 | LYS 143 | LYS 151 | LYS 148 |
| 31 | ASP | ASP 154 | ASP 144 | ASP 152 | ASP 149 |
| 32 | TYR | TYR 155 | TYR 145 | TYR 153 | TYR 150 |
| 33 | PHE | PHE 156 | PHE 146 | PHE 154 | PHE 151 |
| 34 | PRO | PRO 157 | PRO 147 | PRO 155 | PRO 152 |
| 35 | GLU | GLU 158 | GLU 148 | GLU 156 | GLU 153 |
| 36 | PRO | PRO 159 | PRO 149 | PRO 157 | PRO 154 |
| 37 | VAL | VAL 160 | VAL 150 | VAL 158 | VAL 155 |
| 38 | THR | THR 161 | THR 151 | THR 159 | THR 156 |
| 39 | VAL | VAL 162 | VAL 152 | VAL 160 | VAL 157 |
| 40 | SER | SER 163 | SER 153 | SER 161 | SER 158 |
| 41 | TRP | TRP 164 | TRP 154 | TRP 162 | TRP 159 |
| 42 | ASN | ASN 165 | ASN 155 | ASN 163 | ASN 160 |
| 43 | SER | SER 166 | SER 156 | SER 164 | SER 161 |
| 44 | GLY | GLY 167 | GLY 157 | GLY 165 | GLY 162 |
| 45 | ALA | ALA 168 | ALA 158 | ALA 166 | ALA 163 |
| 46 | LEU | LEU 169 | LEU 159 | LEU 167 | LEU 164 |
| 47 | THR | THR 170 | THR 160 | THR 168 | THR 165 |
| 48 | SER | SER 171 | SER 161 | SER 169 | SER 166 |
| 49 | GLY | GLY 172 | GLY 162 | GLY 170 | GLY 167 |
| 50 | VAL | VAL 173 | VAL 163 | VAL 171 | VAL 168 |
| 51 | HIS | HIS 174 | HIS 164 | HIS 172 | HIS 169 |
| 52 | THR | THR 175 | THR 165 | THR 173 | THR 170 |
| 53 | PHE | PHE 176 | PHE 166 | PHE 174 | PHE 171 |
| 54 | PRO | PRO 177 | PRO 167 | PRO 175 | PRO 172 |
| 55 | ALA | ALA 178 | ALA 168 | ALA 176 | ALA 173 |
| 56 | VAL | VAL 179 | VAL 169 | VAL 177 | VAL 174 |
| 57 | LEU | LEU 180 | LEU 170 | LEU 178 | LEU 175 |
| 58 | GLN | GLN 181 | GLN 171 | GLN 179 | GLN 176 |
| 59 | SER | SER 182 | SER 172 | SER 180 | SER 177 |
| 60 | SER | SER 183 | SER 173 | SER 181 | SER 178 |
| 61 | GLY | GLY 184 | GLY 174 | GLY 182 | GLY 179 |
| 62 | LEU | LEU 185 | LEU 175 | LEU 183 | LEU 180 |
| 63 | TYR | TYR 186 | TYR 176 | TYR 184 | TYR 181 |
| 64 | SER | SER 187 | SER 177 | SER 185 | SER 182 |
| 65 | LEU | LEU 188 | LEU 178 | LEU 186 | LEU 183 |
| 66 | SER | SER 189 | SER 179 | SER 187 | SER 184 |
| 67 | SER | SER 190 | SER 180 | SER 188 | SER 185 |
| 68 | VAL | VAL 191 | VAL 181 | VAL 189 | VAL 186 |
| 69 | VAL | VAL 192 | VAL 182 | VAL 190 | VAL 187 |
| 70 | THR | THR 193 | THR 183 | THR 191 | THR 188 |
| 71 | VAL | VAL 194 | VAL 184 | VAL 192 | VAL 189 |
| 72 | PRO | PRO 195 | PRO 185 | PRO 193 | PRO 190 |
| 73 | SER | SER 196 | SER 186 | SER 194 | SER 191 |
| 74 | SER | SER 197 | SER 187 | SER 195 | SER 192 |
| 75 | SER | SER 198 | SER 188 | SER 196 | SER 193 |
| 76 | LEU | LEU 199 | LEU 189 | LEU 197 | LEU 194 |
| 77 | GLY | GLY 200 | GLY 190 | GLY 198 | GLY 195 |
| 78 | THR | THR 201 | THR 191 | THR 199 | THR 196 |
| 79 | GLN | GLN 202 | GLN 192 | GLN 200 | GLN 197 |
| 80 | THR | THR 203 | THR 193 | THR 201 | THR 198 |
| 81 | TYR | TYR 204 | TYR 194 | TYR 202 | TYR 199 |
| 82 | ILE | ILE 205 | ILE 195 | ILE 203 | ILE 200 |
| 83 | CYS | CYS 206 | CYS 196 | CYS 204 | CYS 201 |
| 84 | ASN | ASN 207 | ASN 197 | ASN 205 | ASN 202 |
| 85 | VAL | VAL 208 | VAL 198 | VAL 206 | VAL 203 |
| 86 | ASN | ASN 209 | ASN 199 | ASN 207 | ASN 204 |
| 87 | HIS | HIS 210 | HIS 200 | HIS 208 | HIS 205 |
| 88 | LYS | LYS 211 | LYS 201 | LYS 209 | LYS 206 |
| 89 | PRO | PRO 212 | PRO 202 | PRO 210 | PRO 207 |
| 90 | SER | SER 213 | SER 203 | SER 211 | SER 208 |
| 91 | ASN | ASN 214 | ASN 204 | ASN 212 | ASN 209 |
| 92 | THR | THR 215 | THR 205 | THR 213 | THR 210 |
| 93 | LYS | LYS 216 | LYS 206 | LYS 214 | LYS 211 |
| 94 | VAL | VAL 217 | VAL 207 | VAL 215 | VAL 212 |
| 95 | ASP | ASP 218 | ASP 208 | ASP 216 | ASP 213 |
| 96 | LYS | LYS 219 | LYS 209 | LYS 217 | LYS 214 |
| 97 | LYS | LYS 220 | LYS 210 | LYS 218 | LYS 215 |
| 98 | VAL | VAL 221 | VAL 211 | VAL 219 | VAL 216 |
| 99 | GLU | GLU 222 | GLU 212 | GLU 220 | GLU 217 |
| 100 | PRO | PRO 223 | PRO 213 | | PRO 218 |
| 101 | LYS | LYS 224 | LYS 214 | | LYS 219 |
| 102 | SER | | SER 215 | | SER 220 |
| 103 | CYS | | CYS 216 | | |

Sequence recoding of Cκ

| Recoding | | 1cz8 | 1l7i | 4nyl | 1F8 |
|---|---|---|---|---|---|
| 1 | ARG | ARG 108 | ARG 108 | ARG 108 | ARG 114 |
| 2 | THR | THR 109 | THR 109 | THR 109 | THR 115 |
| 3 | VAL | VAL 110 | VAL 110 | VAL 110 | VAL 116 |
| 4 | ALA | ALA 111 | ALA 111 | ALA 111 | ALA 117 |
| 5 | ALA | ALA 112 | ALA 112 | ALA 112 | ALA 118 |
| 6 | PRO | PRO 113 | PRO 113 | PRO 113 | PRO 119 |
| 7 | SER | SER 114 | SER 114 | SER 114 | SER 120 |
| 8 | VAL | VAL 115 | VAL 115 | VAL 115 | VAL 121 |
| 9 | PHE | PHE 116 | PRE 116 | PHE 116 | PHE 122 |
| 10 | ILE | ILE 117 | ILE 117 | ILE 117 | ILE 123 |
| 11 | PHE | PHE 118 | PRE 118 | PHE 118 | PHE 124 |
| 12 | PRO | PRO 119 | PRO 119 | PRO 119 | PRO 125 |
| 13 | PRO | PRO 120 | PRO 120 | PRO 120 | PRO 126 |
| 14 | SER | SER 121 | SER 121 | SER 121 | SER 127 |
| 15 | ASP | ASP 122 | ASP 122 | ASP 122 | ASP 128 |
| 16 | GLU | GLU 123 | GLU 123 | GLU 123 | GLU 129 |
| 17 | GLN | GLN 124 | GLN 124 | GLN 124 | GLN 130 |
| 18 | LEU | LEU 125 | LEU 125 | LEU 125 | LEU 131 |
| 19 | LYS | LYS 126 | LYS 126 | LYS 126 | LYS 132 |
| 20 | SER | SER 127 | SER 127 | SER 127 | SER 133 |
| 21 | GLY | GLY 128 | GLY 128 | GLY 128 | GLY 134 |
| 22 | THR | THR 129 | THR 129 | THR 129 | THR 135 |
| 23 | ALA | ALA 130 | ALA 130 | ALA 130 | ALA 136 |
| 24 | SER | SER 131 | SER 131 | SER 131 | SER 137 |
| 25 | VAL | VAL 132 | VAL 132 | VAL 132 | VAL 138 |
| 26 | VAL | VAL 133 | VAL 133 | VAL 133 | VAL 139 |
| 27 | CYS | CYS 134 | CYS 134 | CYS 134 | CYS 140 |
| 28 | LEU | LEU 135 | LEU 135 | LEU 135 | LEU 141 |
| 29 | LEU | LEU 136 | LEU 136 | LEU 136 | LEU 142 |
| 30 | ASN | ASN 137 | ASN 137 | ASN 137 | ASN 143 |
| 31 | ASN | ASN 138 | ASN 138 | ASN 138 | ASN 144 |
| 32 | PHE | PHE 139 | PRE 139 | PHE 139 | PHE 145 |
| 33 | TYR | TYR 140 | TYR 140 | TYR 140 | TYR 146 |
| 34 | PRO | PRO 141 | PRO 141 | PRO 141 | PRO 147 |
| 35 | ARG | ARG 142 | ARG 142 | ARG 142 | ARG 148 |
| 36 | GLU | GLU 143 | GLU 143 | GLU 143 | GLU 149 |
| 37 | ALA | ALA 144 | ALA 144 | ALA 144 | ALA 150 |
| 38 | LYS | LYS 145 | LYS 145 | LYS 145 | LYS 151 |
| 39 | VAL | VAL 146 | VAL 146 | VAL 146 | VAL 152 |
| 40 | GLN | GLN 147 | GLN 147 | GLN 147 | GLN 153 |
| 41 | TRP | TRP 148 | TRP 148 | TRP 148 | TRP 154 |
| 42 | LYS | LYS 149 | LYS 149 | LYS 149 | LYS 155 |
| 43 | VAL | VAL 150 | VAL 150 | VAL 150 | VAL 156 |
| 44 | ASP | ASP 151 | ASP 151 | ASP 151 | ASP 157 |
| 45 | ASN | ASN 152 | ASN 152 | ASN 152 | ASN 158 |
| 46 | ALA | ALA 153 | ALA 153 | ALA 153 | ALA 159 |
| 47 | LEU | LEU 154 | LEU 154 | LEU 154 | LEU 160 |
| 48 | GLN | GLN 155 | GLN 155 | GLN 155 | GLN 161 |
| 49 | SER | SER 156 | SER 156 | SER 156 | SER 162 |
| 50 | GLY | GLY 157 | GLY 157 | GLY 157 | GLY 163 |
| 51 | ASN | ASN 158 | ASN 158 | ASN 158 | ASN 164 |
| 52 | SER | SER 159 | SER 159 | SER 159 | SER 165 |
| 53 | GLN | GLN 160 | GLN 160 | GLN 160 | GLN 166 |
| 54 | GLU | GLU 161 | GLU 161 | GLU 161 | GLU 167 |
| 55 | SER | SER 162 | SER 162 | SER 162 | SER 168 |
| 56 | VAL | VAL 163 | VAL 163 | VAL 163 | VAL 169 |
| 57 | THR | THR 164 | THR 164 | THR 164 | THR 170 |
| 58 | GLU | GLU 165 | GLU 165 | GLU 165 | GLU 171 |
| 59 | GLN | GLN 166 | GLN 166 | GLN 166 | GLN 172 |
| 60 | ASP | ASP 167 | ASP 167 | ASP 167 | ASP 173 |
| 61 | SER | SER 168 | SER 168 | SER 168 | SER 174 |
| 62 | LYS | LYS 169 | LYS 169 | LYS 169 | LYS 175 |
| 63 | ASP | ASP 170 | ASP 170 | ASP 170 | ASP 176 |
| 64 | SER | SER 171 | SER 171 | SER 171 | SER 177 |
| 65 | THR | THR 172 | THR 172 | THR 172 | THR 178 |
| 66 | TYR | TYR 173 | TYR 173 | TYR 173 | TYR 179 |
| 67 | SER | SER 174 | SER 174 | SER 174 | SER 180 |
| 68 | LEU | LEU 175 | LEU 175 | LEU 175 | LEU 181 |
| 69 | SER | SER 176 | SER 176 | SER 176 | SER 182 |
| 70 | SER | SER 177 | SER 177 | SER 177 | SER 183 |
| 71 | THR | THR 178 | THR 178 | THR 178 | THR 184 |
| 72 | LEU | LEU 179 | LEU 179 | LEU 179 | LEU 185 |
| 73 | THR | THR 180 | THR 180 | THR 180 | THR 186 |
| 74 | LEU | LEU 181 | LEU 181 | LEU 181 | LEU 187 |
| 75 | SER | SER 182 | SER 182 | SER 182 | SER 188 |
| 76 | LYS | LYS 183 | LYS 183 | LYS 183 | LYS 189 |
| 77 | ALA | ALA 184 | ALA 184 | ALA 184 | ALA 190 |
| 78 | ASP | ASP 185 | ASP 185 | ASP 185 | ASP 191 |
| 79 | TYR | TYR 186 | TYR 186 | TYR 186 | TYR 192 |
| 80 | GLU | GLU 187 | GLU 187 | GLU 187 | GLU 193 |
| 81 | LYS | LYS 188 | LYS 188 | LYS 188 | LYS 194 |
| 82 | HIS | HIS 189 | HIS 189 | HIS 189 | HIS 195 |
| 83 | LYS | LYS 190 | LYS 190 | LYS 190 | LYS 196 |
| 84 | VAL | VAL 191 | VAL 191 | VAL 191 | VAL 197 |
| 85 | TYR | TYR 192 | TYR 192 | TYR 192 | TYR 198 |
| 86 | ALA | ALA 193 | ALA 193 | ALA 193 | ALA 199 |
| 87 | CYS | CYS 194 | CYS 194 | CYS 194 | CYS 200 |
| 88 | GLU | GLU 195 | GLU 195 | GLU 195 | GLU 201 |
| 89 | VAL | VAL 196 | VAL 196 | VAL 196 | VAL 202 |
| 90 | THR | THR 197 | THR 197 | THR 197 | THR 203 |
| 91 | HIS | HIS 198 | HIS 198 | HIS 198 | HIS 204 |
| 92 | GLN | GLN 199 | GLN 199 | GLN 199 | GLN 205 |
| 93 | GLY | GLY 200 | GLY 200 | GLY 200 | GLY 206 |
| 94 | LEU | LEU 201 | LEU 201 | LEU 201 | LEU 207 |
| 95 | SER | SER 202 | SER 202 | SER 202 | SER 208 |
| 96 | SER | SER 203 | SER 203 | SER 203 | SER 209 |
| 97 | PRO | PRO 204 | PRO 204 | PRO 204 | PRO 210 |
| 98 | VAL | VAL 205 | VAL 205 | VAL 205 | VAL 211 |
| 99 | THR | THR 206 | THR 206 | THR 206 | THR 212 |
| 100 | LYS | LYS 207 | LYS 207 | LYS 207 | LYS 213 |
| 101 | SER | SER 208 | SER 208 | SER 208 | SER 214 |
| 102 | PHE | PHE 209 | PRE 209 | PHE 209 | PHE 215 |
| 103 | ASN | ASN 210 | ASN 210 | ASN 210 | ASN 216 |
| 104 | ARG | ARG 211 | ARG 211 | ARG 211 | ARG 217 |
| 105 | GLY | GLY 212 | GLY 212 | | GLY 218 |
| 106 | GLU | GLU 213 | GLU 213 | | GLU 219 |
| 107 | CYS | | CYS 214 | | |

Summary table of top Free energy residues of
1cz8, 4nyl, 1l7i and 1F8

| 1cz8 | | 4nyl | | 1l7i | | 1F8 | | |
|---|---|---|---|---|---|---|---|---|
| 53 | PHE | 53 | PHE | 103 | CYS | 53 | PHE | CH1 |
| 9 | PHE | 9 | PHE | 53 | PHE | 9 | PHE | |
| 11 | LEU | 11 | LEU | 9 | PHE | 11 | LEU | |
| 56 | VAL | 56 | VAL | 101 | LYS | 56 | VAL | |
| 28 | LEU | 28 | LEU | 56 | VAL | 30 | LYS | |
| 51 | HIS | 54 | PRO | 11 | LEU | 96 | LYS | |
| 54 | PRO | 68 | VAL | 54 | PRO | 28 | LEU | |
| 68 | VAL | 24 | ALA | 28 | LEU | 24 | ALA | |
| 24 | ALA | 51 | HIS | 17 | SER | 68 | VAL | |
| | | | | 68 | VAL | 54 | PRO | |
| | | | | 24 | ALA | | | |
| | | | | 96 | LYS | | | |
| 11 | PHE | 11 | PHE | 11 | PHE | 11 | PHE | Cκ |
| 9 | PHE | 9 | PHE | 9 | PHE | 9 | PHE | |
| 28 | LEU | 28 | LEU | 100 | LYS | 100 | LYS | |
| 26 | VAL | 57 | THR | 57 | THR | 57 | THR | |
| 53 | GLN | 26 | VAL | 107 | CYS | 28 | LEU | |
| 14 | SER | 53 | GLN | 28 | LEU | 26 | VAL | |
| 30 | ASN | 30 | ASN | 26 | VAL | 53 | GLN | |
| | | | | 53 | GLN | | | |
| | | | | 12 | PRO | | | |

Bold: Unique residues
Underlined: Low homologous residues
No marking: Conserved residues

Residues with most stabilizing effects

| | | | |
|---|---|---|---|
| 1cz8 | 4nyl | 1l7i | 1F8 |

CH

| | | | | | |
|---|---|---|---|---|---|
| 9 | PHE | 9 | PHE | 9 | PHE |
| 51 | PHE | 11 | LEU | | |
| 53 | PHE | 53 | PHE | 53 | PHE |
| 54 | PRO | 103 | CYS | | |

CK

| | | | | | | | |
|---|---|---|---|---|---|---|---|
| | | 9 | PHE | 9 | PHE | 9 | PHE |
| 11 | PHE | 11 | PHE | 11 | PHE | 11 | PHE |

Five important interface residues for Cκ and CH1 interaction
(based on structure and free energy)

| CH1 | | Cκ | |
|---|---|---|---|
| Position | Residue | Position | Residue |
| 9 | PHE | 17 | GLN |
| 11 | LEU | 11, 26 | PHE, VAL |
| 24 | ALA | 9, 11 | PHE |
| 51 | HIS | 31 | ASN |
| 53 | PHE | 69 | SER |

Note:
Salt bridge residues are excluded

### Example 2: Discovery of Important Interface Residues for Cκ and CH1 Interaction

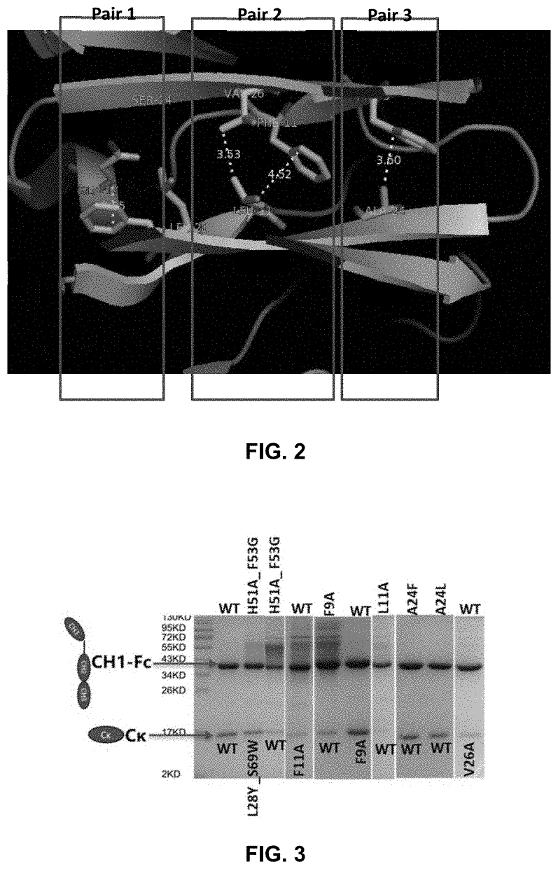

Based on the interface analysis of Cκ and CH1, this example summarized the top important interface residues for Cκ and CH1 interaction (see FIG. 2 and Table 4 below).

TABLE 4
Residue Pairs Impacting Cκ/CH1 Interaction

| CH1 | | Cκ | | |
|---|---|---|---|---|
| Position | Residue | Position | Residue | Pair No. |
| 9 | PHE | 17 | GLN | 1 |
| 11 | LEU | 11, 26 | PHE, VAL | 2 |
| 24 | ALA | 9, 11 | PHE | 3 |
| 51 | HIS | 31 | ASN | 4 |
| 53 | PHE | 69 | SER | 5 |

Note:
Salt bridge residues are excluded

From the table above, alanine or tryptophan single mutations were used to test each interface residue. IgG(-Fv) without VH and VL was constructed and expressed for Ala and Trp screening. The mutation list is listed as below.

Alanine screening

| Name | Description | Cκ | CH1 |
|---|---|---|---|
| Cκ/CH1_001 | Cκ/CH1 | WT | WT |
| Cκ/CH1_002 | Cκ_L28Y_S69W/CH1_H51A_F53G | L28Y_S69W | H51A_F53G |
| Cκ/CH1_003 | Cκ/CH1_H51A_F53G | WT | H51A_F53G |
| Cκ/CH1_004 | Cκ/CH1_D31K_F53T_V68F | WT | D31K_F53T_V68F |
| Cκ/CH1_005 | Cκ/CH1_F9A_F53A | WT | F9A_F53A |
| Cκ/CH1_006 | Cκ_F9G_F11A_K100A/CH1 | F9G_F11A_K100A | WT |
| Cκ/CH1_007 | Cκ_F11A/CH1 | F11A | WT |
| Cκ/CH1_008 | Cκ_F9A_F11A/CH1 | F9A_F11A | WT |
| Cκ/CH1_009 | Cκ_F11A_K100A/CH1 | F11A_K100A | WT |
| Cκ/CH1_010 | Cκ_F9A_K100A/CH1 | F9A_K100A | WT |
| Cκ/CH1_011 | Cκ/CH1_F9A_L11A | WT | F9A_L11A |
| Cκ/CH1_012 | Cκ/CH1_L11A_F53A | WT | L11A_F53A |
| Cκ/CH1_013 | Cκ/CH1_F9A | WT | F9A |
| Cκ/CH1_014 | Cκ/CH1_L11A | WT | L11A |
| Cκ/CH1_015 | Cκ_F9A/CH1 | F9A | WT |
| Cκ/CH1_016 | Cκ_F9A_F11M/CH1 | F9A_F11M | WT |
| Cκ/CH1_017 | Cκ/CH1_A24F | WT | A24F |
| Cκ/CH1_018 | Cκ/CH1_A24L | WT | A24L |
| Cκ/CH1_019 | Cκ_F9A_F11A/CH1_A24F | F9A_F11A | A24F |
| Cκ/CH1_020 | Cκ_F9A_F11A/CH1_A24L | F9A_F11A | A24L |
| Cκ/CH1_021 | Cκ_F9A_F11M/CH1_A24F | F9A_F11M | A24F |
| Cκ/CH1_022 | Cκ_F9A_F11M/CH1_A24L | F9A_F11M | A24L |
| Cκ/CH1_023 | Cκ_V26A/CH1 | V26A | WT |
| Cκ/CH1_024 | Cκ_V26A_F11A/CH1 | V26A_F11A | WT |
| Cκ/CH1_025 | Cκ/CH1_L11F_L28G | WT | L11F_L28G |
| Cκ/CH1_026 | Cκ_V26A/CH1_L11F_L28G | V26A | L11F_L28G |
| Cκ/CH1_027 | Cκ_V26A_F11A/CH1_L11F_L28G | V26A_F11A | L11F_L28G |

Tryptophan screening

| Name | Description | Cκ | CH1 |
|---|---|---|---|
| Cκ/CH1_028 | Cκ/CH1_A24W | WT | A24W |
| Cκ/CH1_029 | Cκ/CH1_L11F | WT | L11F |
| Cκ/CH1_030 | Cκ/CH1_L11W | WT | L11W |
| Cκ/CH1_031 | Cκ_F9A_F11A/CH1_L11F_A24F | F9A_F11A | L11F_A24F |
| Cκ/CH1_032 | Cκ_V26W/CH1 | V26W | WT |

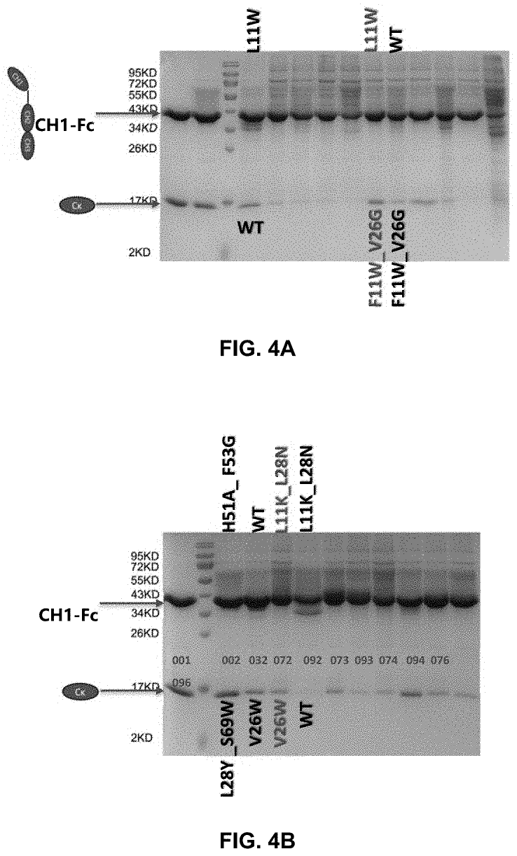

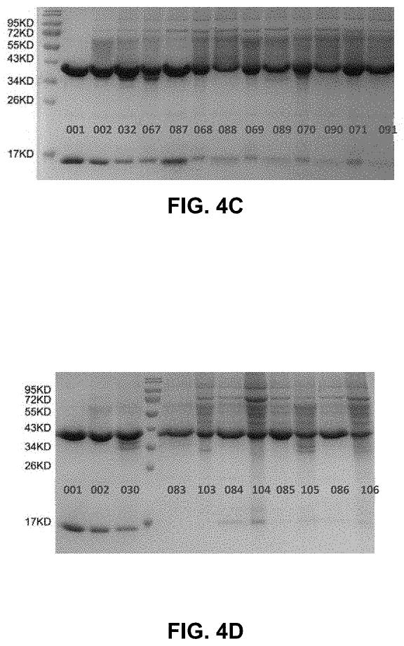

As shown in the SDS-PAGE image of FIG. 3, for Pair 2 (Cκ_F11_V26 and CH1_L11), the two mutants Cκ_F11A/CH1 and Cκ_V26A/CH1 greatly interrupted the interaction of Cκ and CH1; the two mutants Cκ_V26W/CH1 and Cκ/CH1_L11W also disrupted the interaction (FIG. 4). Mutations Cκ/CH1_L11A and Cκ/CH1_F9A (from Pair 1) also disrupted the interaction. Mutants Cκ_F9A/CH1, Cκ/CH1_A24F and Cκ/CH1_A24L, by contrast, did not affect the interaction of Cκ and CH1. This suggests that Pair 3 (Cκ_F9 and CH1_A24) is not important for the binding of Cκ and CH1.

| Pair No. | CH1 | Cκ | Important |
|---|---|---|---|
| Pair 1 | Phe9 | Gln17 | Yes |
| Pair 2 | Leu11 | Phe11, Val26 | Yes |
| Pair 3 | Ala24 | Phe9 | No |

### Example 3: Mutation Pair Development for Pair 1 by Discovery Studio

Upon identification of residue pairs that are important for maintaining the interaction between Cκ and CH1, this example tested mutation pairs that establish new interactions. The rationale of this development is that: mutant Cκ can show good binding to mutant CH1; but mutant Cκ does not bind or weakly bind to wild type CH1 and mutant CH1 show weak or no binding to wild type Cκ.

### Mutation Development for Pair 1

The residues in Pair 1 are Cκ_Q17 and CH1_F9 (Table 4). These mutations of Cκ/CH1_033 to 050 were designed and analyzed by the inventors. Cκ/CH1_051-066 mutation pairs were developed by a software program, Discovery Studio (DS), to design random mutations for this site. It generated eight pairs for Cκ_Q17 and CH1_F9 as listed below.

| | Mutation | | | | | |
|---|---|---|---|---|---|---|
| Mutation | energy | Effect | VDW | Electro-static | Entropy | Non-polar |
| H:PHE9>ILE.L:GLN17>HIS | 0.03 | Neutral | 0.15 | 0.03 | −0.07 | 0 |
| H:PHE9>HIS.L:GLN17>ARG | 0.07 | Neutral | −1.42 | 1.4 | 0.09 | 0 |
| H:PHE9>LYS.L:GLN17>ASP | 0.09 | Neutral | 2.15 | −3.23 | 0.72 | 0 |
| H:PHE9>HIS.L:GLN17>HIS | 0.19 | Neutral | 0.16 | −0.07 | 0.17 | 0 |
| H:PHE9>PRO.L:GLN17>ARG | 0.25 | Neutral | 0.13 | 1.33 | −0.55 | 0 |
| H:PHE9>MET.L:GLN17>HIS | 0.29 | Neutral | 0.12 | 0.09 | 0.21 | 0 |
| H:PHE9>GLN.L:GLN17>ARG | 0.3 | Neutral | −0.42 | 1.89 | −0.49 | 0 |
| H:PHE9>GLN.L:GLN17>HIS | 0.33 | Neutral | −0.46 | −0.12 | 0.7 | 0 |

Notes:
Mutation energy: energy difference after mutation; low value means more stable;
VDW: Van der Waals

| | | | |
|---|---|---|---|
| Cκ/CH1_033 | Cκ_Q17R/CH1 | Q17R | WT |
| Cκ/CH1_034 | Cκ_Q17K/CH1 | Q17K | WT |
| Cκ/CH1_035 | Cκ_Q17D/CH1 | Q17D | WT |
| Cκ/CH1_036 | Cκ_Q17E/CH1 | Q17E | WT |
| Cκ/CH1_037 | Cκ/CH1_F9R | WT | F9R |
| Cκ/CH1_038 | Cκ/CH1_F9K | WT | F9K |
| Cκ/CH1_039 | Cκ/CH1_F9D | WT | F9D |
| Cκ/CH1_040 | Cκ/CH1_F9E | WT | F9E |
| Cκ/CH1_041 | Cκ_F11E_V26A/CH1_L11R | F11E_V26A | L11R |
| Cκ/CH1_042 | Cκ_Q17K_F11K_V26A/CH1_F9E_L11E | Q17K_F11K_V26A | F9E_L11E |
| Cκ/CH1_043 | Cκ_Q17R/CH1_F9D | Cκ_Q17R | CH1_F9D |
| Cκ/CH1_044 | Cκ_Q17K/CH1_F9D | Cκ_Q17K | CH1_F9D |
| Cκ/CH1_045 | Cκ_Q17R/CH1_F9E | Cκ_Q17R | CH1_F9E |
| Cκ/CH1_046 | Cκ_Q17K/CH1_F9E | Cκ_Q17K | CH1_F9E |
| Cκ/CH1_047 | Cκ_Q17D/CH1_F9R | Cκ_Q17D | CH1_F9R |
| Cκ/CH1_048 | Cκ_Q17D/CH1_F9K | Cκ_Q17D | CH1_F9K |
| Cκ/CH1_049 | Cκ/CH1_F9D_L11A | Cκ | CH1_F9D_L11A |
| Cκ/CH1_050 | Cκ_Q17K/CH1_F9D_L11A | Cκ_Q17K | CH1_F9D_L11A |
| Cκ/CH1_051 | Cκ_Q17H/CH1_F9I | Cκ_Q17H | CH1_F9I |
| Cκ/CH1_052 | Cκ_Q17R/CH1_F9H | Cκ_Q17R | CH1_F9H |
| Cκ/CH1_053 | Cκ_Q17H/CH1_F9H | Cκ_Q17H | CH1_F9H |
| Cκ/CH1_054 | Cκ_Q17R/CH1_F9P | Cκ_Q17R | CH1_F9P |
| Cκ/CH1_055 | Cκ_Q17D/CH1_F9H | Cκ_Q17D | CH1_F9H |
| Cκ/CH1_056 | Cκ_Q17I/CH1_F9H | Cκ_Q17I | CH1_F9H |
| Cκ/CH1_057 | Cκ_Q17H/CH1_F9M | Cκ_Q17H | CH1_F9M |
| Cκ/CH1_058 | Cκ_Q17R/CH1_F9Q | Cκ_Q17R | CH1_F9Q |
| Cκ/CH1_059 | Cκ_Q17H/CH1_F9Q | Cκ_Q17H | CH1_F9Q |
| Cκ/CH1_060 | Cκ_Q17H/CH1 | Cκ_Q17H | CH1 |
| Cκ/CH1_061 | Cκ_Q17I/CH1 | Cκ_Q17I | CH1 |
| Cκ/CH1_062 | Cκ/CH1_F9I | Cκ | CH1_F9I |
| Cκ/CH1_063 | Cκ/CH1_F9H | Cκ | CH1_F9H |
| Cκ/CH1_064 | Cκ/CH1_F9P | Cκ | CH1_F9P |
| Cκ/CH1_065 | Cκ/CH1_F9M | Cκ | CH1_F9M |
| Cκ/CH1_066 | Cκ/CH1_F9Q | Cκ | CH1_F9Q |

Two good mutation pairs are listed below:

| Mutation ID | Position Numbering | Kabat Numbering |
|---|---|---|
| Cκ/CH1_043 | Cκ_Q17R/CH1_F9D | Cκ_Q124R/CH1_F122D |
| Cκ/CH1_044 | Cκ_Q17K/CH1_F9D | Cκ_Q124K/CH1_F122D |

### Example 4: Mutation Pair Development for Pair 2 by Discovery Studio

For Pair 2, alanine/tryptophan single mutations were tested for each interface residue. IgG(-Fv) without VH and VL was constructed and expressed for Ala and Trp screening. This example used Discovery Studio to design random mutations for this site.

Three good mutation pairs are Cκ/CH1_072, Cκ/CH1_079 and Cκ/CH1_107 listed below:

| Mutation ID | Position Numbering | Kabat Numbering |
|---|---|---|
| Cκ/CH1_072 | Cκ_V26W/ | Cκ_V133W/ |
| | CH1_L11K_L28N | CH1_L124K_L141N |
| Cκ/CH1_079 | Cκ_F11W_V26G/ | Cκ_F118W_V133G/ |
| | CH1_L11W | CH1_L124W |
| Cκ/CH1_107 | Cκ_V26W/CH1_L11W | Cκ_V133W/CH1_L124W |

### Mutation Development for Pair 2

The important residues for Pair 2 are Cκ_F11_V26 and CH1_L11_L28 (see Table 4). The strategy of mutation development for this hot spot is to fix mutation V26W or L11W. This example also tested introducing saturated point mutations for Cκ_F11_V26 and CH1_L11_L28; then applying DS to calculate all potent mutations.

Strategy I: with fixed mutation V26W, random point mutations were introduced into CH1_L11_L28; then DS software was used to generate some mutation pairs for this site. Some preferable mutation pairs are listed as below.

| | Mutation | | | Electro- | | |
|---|---|---|---|---|---|---|
| Mutation | energy | Effect | VDW | static | Entropy | Non-polar |
| H:LEU11>VAL.L:VAL26>TRP | −0.14 | Neutral | −0.31 | 0.11 | −0.04 | 0 |
| H:LEU11>ASN.L:VAL26>TRP | −0.08 | Neutral | −0.56 | 0.29 | 0.06 | 0 |
| H:LEU11>MET.L:VAL26>TRP | −0.03 | Neutral | −0.46 | 0.32 | 0.04 | 0 |
| H:LEU11>ILE.L:VAL26>TRP | 0.18 | Neutral | −0.01 | 0.3 | 0.04 | 0 |
| H:LEU11>SER.L:VAL26>TRP | 0.31 | Neutral | −0.83 | 0.58 | 0.49 | 0 |
| H:LEU11>GLU.L:VAL26>TRP | 0.38 | Neutral | −1.76 | 2.45 | 0.04 | 0 |
| H:LEU11>GLY.L:VAL26>TRP | 0.41 | Neutral | 0.16 | 0.18 | 0.27 | 0 |

| | Mutation | | | Electro- | | Non- |
|---|---|---|---|---|---|---|
| Mutation | energy | Effect | VDW | static | Entropy | polar |
| H:LEU28>SER.L:VAL26>TRP | −0.54 | Stabilizing | −2.58 | 0.34 | 0.66 | 0 |
| H:LEU28>GLU.L:VAL26>TRP | −0.5 | Neutral | −4.5 | 3.13 | 0.21 | 0 |
| H:LEU28>ASN.L:VAL26>TRP | −0.43 | Neutral | −2.26 | 0.37 | 0.58 | 0 |
| H:LEU28>CYS.L:VAL26>TRP | −0.21 | Neutral | −1.49 | 0.12 | 0.54 | 0 |
| H:LEU28>THR.L:VAL26>TRP | −0.09 | Neutral | −1.22 | 0.3 | 0.42 | 0 |
| H:LEU28>VAL.L:VAL26>TRP | −0.06 | Neutral | −1.14 | 0.16 | 0.49 | 0 |
| H:LEU28>ALA.L:VAL26>TRP | 0.1 | Neutral | −1.04 | 0.12 | 0.64 | 0 |
| H:LEU28>ASP.L:VAL26>TRP | 0.38 | Neutral | −2.6 | 2.36 | 0.57 | 0 |

| | Mutation | | | Electro- | | Non- |
|---|---|---|---|---|---|---|
| Mutation | energy | Effect | VDW | static | Entropy | polar |
| H:LEU11>ILE.H:LEU28>PHE.L:VAL26>TRP | −2.85 | Stabilizing | −6.21 | 0.1 | 0.23 | 0 |
| H:LEU11>ARG.H:LEU28>PRO.L:VAL26>TRP | −2.38 | Stabilizing | −7.66 | 0.45 | 1.39 | 0 |
| H:LEU11>ILE.H:LEU28>GLN.L:VAL26>TRP | −1.86 | Stabilizing | −4.73 | 0.48 | 0.3 | 0 |
| H:LEU11>ARG.H:LEU28>GLY.L:VAL26>TRP | −1.64 | Stabilizing | −6.13 | 0.44 | 1.37 | 0 |
| H:LEU11>ARG.H:LEU28>ASP.L:VAL26>TRP | −1.46 | Stabilizing | −7.85 | 2.67 | 1.29 | 0 |
| H:LEU11>LYS.H:LEU28>ASN.L:VAL26>TRP | −1.33 | Stabilizing | −5.94 | 1.42 | 1.06 | 0 |
| H:LEU11>THR.H:LEU28>HIS.L:VAL26>TRP | −1.32 | Stabilizing | −4.18 | 0.55 | 0.56 | 0 |
| H:LEU11>LEU.H:LEU28>THR.L:VAL26>TRP | −1.17 | Stabilizing | −3.35 | 0.42 | 0.33 | 0 |
| H:LEU11>ALA.H:LEU28>ARG.L:VAL26>TRP | −1.16 | Stabilizing | −4.55 | −0.57 | 1.59 | 0 |
| H:LEU11>GLU.H:LEU28>GLN.L:VAL26>TRP | −1.12 | Stabilizing | −5.43 | 2.65 | 0.31 | 0 |

Strategy 2: with fixed mutation L11W, random point mutations were introduced into Cκ_F11V26; then the DS software was used to generate some mutation pairs for this site. Some preferable mutation pairs are listed as below.

| | Mutation | | | Electro- | | Non- |
|---|---|---|---|---|---|---|
| Mutation | energy | Effect | VDW | static | Entropy | polar |
| H:LEU11>TRP.L:VAL26>LEU | −2.24 | Stabilizing | −4.78 | 0.32 | −0.01 | 0 |
| H:LEU11>TRP.L:VAL26>MET | −1.89 | Stabilizing | −4.2 | 0.19 | 0.13 | 0 |
| H:LEU11>TRP.L:VAL26>TRP | −1.38 | Stabilizing | −3.01 | 0.58 | −0.19 | 0 |
| H:LEU11>TRP.L:VAL26>GLU | −1.21 | Stabilizing | −3.88 | 0.87 | 0.34 | 0 |
| H:LEU11>TRP.L:VAL26>LYS | −0.9 | Stabilizing | −5.72 | 3.44 | 0.27 | 0 |
| H:LEU11>TRP.L:VAL26>CYS | −0.84 | Stabilizing | −1.95 | 0.12 | 0.09 | 0 |
| H:LEU11>TRP.L:VAL26>SER | −0.68 | Stabilizing | −2.65 | 0.42 | 0.49 | 0 |
| H:LEU11>TRP.L:VAL26>ALA | −0.6 | Stabilizing | −1.65 | 0.02 | 0.25 | 0 |
| H:LEU11>TRP.L:VAL26>GLY | −0.26 | Neutral | −1.66 | 0.16 | 0.56 | 0 |
| H:LEU11>TRP.L:VAL26>PRO | −0.19 | Neutral | −0.91 | 0.1 | 0.25 | 0 |

| | Mutation | | | Electro- | | Non- |
|---|---|---|---|---|---|---|
| Mutation | energy | Effect | VDW | static | Entropy | polar |
| H:LEU11>TRP.L: | −0.3 | Neutral | −1.46 | 0.14 | 0.41 | 0 |
| PHE11>HIS | | | | | | |

| | Mutation | | | Electro- | | Non- |
|---|---|---|---|---|---|---|
| Mutation | energy | Effect | VDW | static | Entropy | polar |
| H:LEU11>TRP.L:PHE11>TRP.L:VAL26> | −1.94 | Stabilizing | −7.43 | 3.17 | 0.22 | 0 |
| LYS | | | | | | |
| H:LEU11>TRP.L:PHE11>HIS.L:VAL26> | −1.68 | Stabilizing | −7.35 | 2.56 | 0.81 | 0 |
| ARG | | | | | | |
| H:LEU11>TRP.L:PHE11>TRP.L:VAL26> | −1.64 | Stabilizing | −4.45 | 0.07 | 0.63 | 0 |
| GLY | | | | | | |
| H:LEU11>TRP.L:PHE11>HIS.L:VAL26> | −1.58 | Stabilizing | −4 | 0.3 | 0.31 | 0 |
| LEU | | | | | | |
| H:LEU11>TRP.L:PHE11>ARG.L: | −1.56 | Stabilizing | −6.43 | 2.37 | 0.54 | 0 |
| VAL26>TYR | | | | | | |
| H:LEU11>TRP.L:PHE11>ARG.L: | −1.34 | Stabilizing | −6.03 | 1.87 | 0.84 | 0 |
| VAL26>GLU | | | | | | |
| H:LEU11>TRP.L:PHE11>HIS.L:VAL26> | −1.25 | Stabilizing | −3.3 | 0.07 | 0.42 | 0 |
| MET | | | | | | |
| H:LEU11>TRP.L:PHE11>HIS.L:VAL26> | −1.21 | Stabilizing | −3.06 | 0.33 | 0.18 | 0 |
| TRP | | | | | | |
| H:LEU11>TRP.L:PHE11>LEU.L: | −1.16 | Stabilizing | −5.72 | 2.42 | 0.56 | 0 |
| VAL26>ARG | | | | | | |
| H:LEU11>TRP.L:PHE11>ARG.L: | −1.09 | Stabilizing | −5.83 | 2.2 | 0.82 | 0 |
| VAL26>LEU | | | | | | |

Strategy 3: saturated point mutations were introduced for Cκ_F11_V26 and CH1_L11_L28; then DS was used to calculate all potent mutations. It generated 23 preferable mutation pairs listed below.

| | Mutation | | | Electro- | | Non- |
|---|---|---|---|---|---|---|
| Mutation | energy | Effect | VDW | static | Entropy | polar |
| H:LEU11>VAL.L:VAL26>TRP | −0.14 | Neutral | −0.31 | 0.11 | −0.04 | 0 |
| H:LEU11>ASN.L:VAL26>TRP | −0.08 | Neutral | −0.56 | 0.29 | 0.06 | 0 |
| H:LEU11>MET.L:VAL26>TRP | −0.03 | Neutral | −0.46 | 0.32 | 0.04 | 0 |
| H:LEU11>MET.L:VAL26>GLU | 0.14 | Neutral | −0.04 | −0.17 | 0.28 | 0 |
| H:LEU11>ASN.L:VAL26>GLU | 0.16 | Neutral | −0.65 | 0.14 | 0.47 | 0 |
| H:LEU11>ILE.L:VAL26>TRP | 0.18 | Neutral | −0.01 | 0.3 | 0.04 | 0 |
| H:LEU11>PRO.L:VAL26>GLU | 0.28 | Neutral | 0.39 | −0.52 | 0.39 | 0 |
| H:LEU11>MET.L:VAL26>LEU | 0.3 | Neutral | 0.78 | 0.05 | −0.13 | 0 |
| H:LEU11>SER.L:VAL26>TRP | 0.31 | Neutral | −0.83 | 0.58 | 0.49 | 0 |
| H:LEU11>VAL.L:VAL26>GLU | 0.34 | Neutral | 0.22 | −0.32 | 0.44 | 0 |
| H:LEU11>GLU.L:VAL26>TRP | 0.38 | Neutral | −1.76 | 2.45 | 0.04 | 0 |
| H:LEU11>GLY.L:VAL26>TRP | 0.41 | Neutral | 0.16 | 0.18 | 0.27 | 0 |
| H:LEU11>ILE.L:VAL26>GLU | 0.43 | Neutral | 0.38 | −0.22 | 0.4 | 0 |
| H:LEU11>MET.L:VAL26>MET | 0.44 | Neutral | 0.91 | 0.01 | −0.02 | 0 |
| H:LEU28>SER.L:VAL26>TRP | −0.54 | Stabilizing | −2.58 | 0.34 | 0.66 | 0 |
| H:LEU28>GLU.L:VAL26>TRP | −0.5 | Neutral | −4.5 | 3.13 | 0.21 | 0 |
| H:LEU28>ASN.L:VAL26>TRP | −0.43 | Neutral | −2.26 | 0.37 | 0.58 | 0 |
| H:LEU28>CYS.L:VAL26>TRP | −0.21 | Neutral | −1.49 | 0.12 | 0.54 | 0 |
| H:LEU28>THR.L:VAL26>TRP | −0.09 | Neutral | −1.22 | 0.3 | 0.42 | 0 |
| H:LEU28>VAL.L:VAL26>TRP | −0.06 | Neutral | −1.14 | 0.16 | 0.49 | 0 |
| H:LEU28>ALA.L:VAL26>TRP | 0.1 | Neutral | −1.04 | 0.12 | 0.64 | 0 |
| H:LEU28>ASP.L:VAL26>TRP | 0.38 | Neutral | −2.6 | 2.36 | 0.57 | 0 |
| H:LEU28>VAL.L:VAL26>GLU | 0.42 | Neutral | −0.19 | −0.07 | 0.62 | 0 |

Based on the above the mutation pairs, for Pair 1, all of the mutation pairs were analyzed by SDS-PAGE (Reduced and Non-Reduced, FIG. 4A-D); for pair 2, some potent mutation pairs with the lowest free energy were chosen for analysis. Among the all mutation pairs, three mutation pairs Cκ/CH1_107 are more potent. The results can be comparable to published mutation pair. IgG(-Fv) without VH and VL was constructed and expressed for each mutation pair. Mutation list is listed as below.

Three good mutation pairs are Cκ/CH1_072, Cκ/CH1_079 and Cκ/CH1_107 listed below:

| | |
|---|---|
| Cκ/CH1_072 | Cκ_V26W/CH1_L11K_L28N |
| Cκ/CH1_079 | Cκ_F11W_V26G/CH1_L1W |
| Cκ/CH1_107 | Cκ_V26W/CH1_L11W |

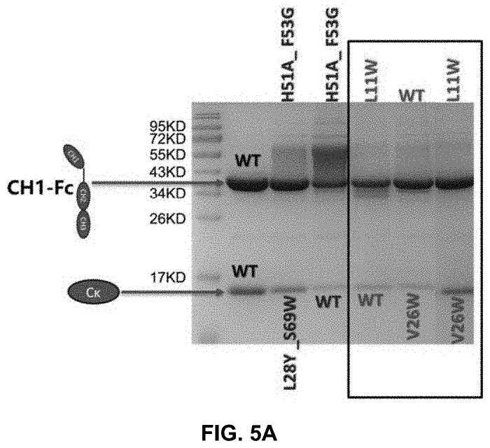

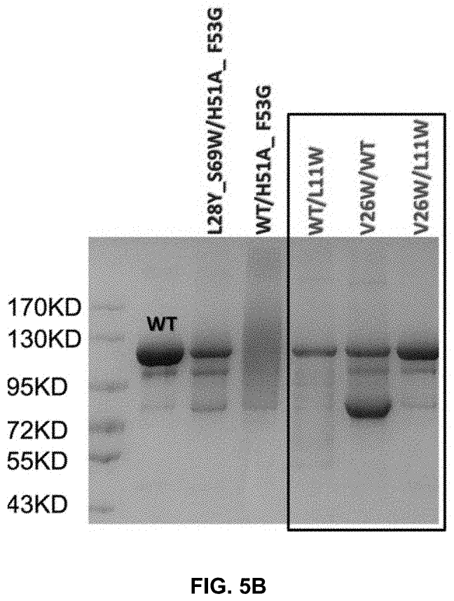

As shown in the SDS-PAGE gel pictures in FIG. 5A-5B, mutation pair Cκ_V26W/CH1_L11W re-established binding between Cκ and CH1 (Cκ_L28Y_S69W/CH1_H51A_F53G was used as control).

| | | | |
|---|---|---|---|
| Cκ/CH1_067 | Cκ_V26W/CH1_L11I_L28F | Cκ_V26W | CH1_L11I_L28F |
| Cκ/CH1_068 | Cκ_V26W/CH1_L11R_L28P | Cκ_V26W | CH1_L11R_L28P |
| Cκ/CH1_069 | Cκ_V26W/CH1_L11I_L28Q | Cκ_V26W | CH1_L11I_L28Q |
| Cκ/CH1_070 | Cκ_V26W/CH1_L11R_L28G | Cκ_V26W | CH1_L11R_L28G |
| Cκ/CH1_071 | Cκ_V26W/CH1_L11R_L28D | Cκ_V26W | CH1_L11R_L28D |
| Cκ/CH1_072 | Cκ_V26W/CH1_L11K_L28N | Cκ_V26W | CH1_L11K_L28N |
| Cκ/CH1_073 | Cκ_V26W/CH1_L11T_L28H | Cκ_V26W | CH1_L11T_L28H |
| Cκ/CH1_074 | Cκ_V26W/CH1_L28T | Cκ_V26W | CH1_L28T |
| Cκ/CH1_075 | Cκ_V26W/CH1_L11A_L28R | Cκ_V26W | CH1_L11A_L28R |
| Cκ/CH1_076 | Cκ_V26W/CH1_L11E_L28Q | Cκ_V26W | CH1_L11E_L28Q |
| Cκ/CH1_077 | Cκ_F11W_V26K/CH1_L11W | Cκ_F11W_V26K | CH1_L11W |
| Cκ/CH1_078 | Cκ_F11H_V26R/CH1_L11W | Cκ_F11H_V26R | CH1_L11W |
| Cκ/CH1_079 | Cκ_F11W_V26G/CH1_L11W | Cκ_F11W_V26G | CH1_L11W |
| Cκ/CH1_080 | Cκ_F11H_V26L/CH1_L11W | Cκ_F11H_V26L | CH1_L11W |
| Cκ/CH1_081 | Cκ_F11R_V26Y/CH1_L11W | Cκ_F11R_V26Y | CH1_L11W |
| Cκ/CH1_082 | Cκ_F11R_V26E/CH1_L11W | Cκ_F11R_V26E | CH1_L11W |
| Cκ/CH1_083 | Cκ_F11H_V26M/CH1_L11W | Cκ_F11H_V26M | CH1_L11W |
| Cκ/CH1_084 | Cκ_F11H_V26W/CH1_L11W | Cκ_F11H_V26W | CH1_L11W |
| Cκ/CH1_085 | Cκ_F11L_V26R/CH1_L11W | Cκ_F11L_V26R | CH1_L11W |
| Cκ/CH1_086 | Cκ_F11R_V26L/CH1_L11W | Cκ_F11R_V26L | CH1_L11W |
| Cκ/CH1_087 | Cκ/CH1_L11I_L28F | Cκ | CH1_L11I_L28F |
| Cκ/CH1_088 | Cκ/CH1_L11R_L28P | Cκ | CH1_L11R_L28P |
| Cκ/CH1_089 | Cκ/CH1_L11I_L28Q | Cκ | CH1_L11I_L28Q |
| Cκ/CH1_090 | Cκ/CH1_L11R_L28G | Cκ | CH1_L11R_L28G |
| Cκ/CH1_091 | Cκ/CH1_L11R_L28D | Cκ | CH1_L11R_L28D |
| Cκ/CH1_092 | Cκ/CH1_Ll1K_L28N | Cκ | CH1_L11K_L28N |
| Cκ/CH1_093 | Cκ/CH1_L11T_L28H | Cκ | CH1_L11T_L28H |
| Cκ/CH1_094 | Cκ/CH1_L28T | Cκ | CH1_L28T |
| Cκ/CH1_095 | Cκ/CH1_L11A_L28R | Cκ | CH1_L11A_L28R |
| Cκ/CH1_096 | Cκ/CH1_L11E_L28Q | Cκ | CH1_L11E_L28Q |
| Cκ/CH1_097 | Cκ_F11W_V26K/CH1 | Cκ_F11W_V26K | CH1 |
| Cκ/CH1_098 | Cκ_F11H_V26R/CH1 | Cκ_F11H_V26R | CH1 |
| Cκ/CH1_099 | Cκ_F11W_V26G/CH1 | Cκ_F11W_V26G | CH1 |
| Cκ/CH1_100 | Cκ_F11H_V26L/CH1 | Cκ_F11H_V26L | CH1 |
| Cκ/CH1_101 | Cκ_F11R_V26Y/CH1 | Cκ_F11R_V26Y | CH1 |
| Cκ/CH1_102 | Cκ_F11R_V26E/CH1 | Cκ_F11R_V26E | CH1 |
| Cκ/CH1_103 | Cκ_F11H_V26M/CH1 | Cκ_F11H_V26M | CH1 |
| Cκ/CH1_104 | Cκ_F11H_V26W/CH1 | Cκ_F11H_V26W | CH1 |
| Cκ/CH1_105 | Cκ_F11L_V26R/CH1 | Cκ_F11L_V26R | CH1 |
| Cκ/CH1_106 | Cκ_F11R_V26L/CH1 | Cκ_F11R_V26L | CH1 |
| Cκ/CH1_107 | Cκ_V26W/CH1_L11W | Cκ_V26W | CH1_L11W |

### Example 5: Mutation Pair Cκ_V26W/CH1_L11W Improvement by Discovery Studio

Strategy 4: With fixed mutation Cκ_V26W and CH1_L11W, saturated point mutations were introduced for Cκ_F11 and CH1_L28; then DS was used to calculate all potent mutations. It generated 23 preferable mutation pairs listed below.

| | mutation | | | ELECTRO- | | non- | double |
|---|---|---|---|---|---|---|---|
| mutation | energy | effect | VDW | STATIC | ENTROPY | polar | mutation |
| H:LEU11>TRP.L:PHE11>HIS.L: | −1.21 | STABILIZING | −3.06 | 0.33 | 0.18 | 0 | 1.17 |
| VAL26>TRP | | | | | | | |
| H:LEU11>TRP.L:PHE11>ALA. | −0.27 | NEUTRAL | −0.35 | −0.18 | 0 | 0 | 4.52 |
| L:VAL26>TRP | | | | | | | |
| H:LEU11>TRP.H:LEU28>ARG. | −0.78 | STABILIZING | −3.18 | 0.44 | 0.67 | 0 | 0.72 |
| L:VAL26>TRP | | | | | | | |
| H:LEU11>TRP.H:LEU28>PRO. | 0.42 | NEUTRAL | −0.63 | 0.6 | 0.49 | 0 | 2.86 |
| L:VAL26>TRP | | | | | | | |
| H:LEU11>TRP.H:LEU28>ARG. | −0.34 | NEUTRAL | −2.12 | −0.22 | 0.94 | 0 | H: 0.72 |
| L:PHE11>CYS.L:VAL26>TRP | | | | | | | L: 4.06 |
| H:LEU11>TRP.H:LEU28>ARG. | −0.25 | NEUTRAL | −1.76 | 0.41 | 0.48 | 0 | H: 0.72 |
| L:PHE11>ILE.L:VAL26>TRP | | | | | | | L: 2.87 |
| H:LEU11>TRP.H:LEU28>ARG. | −0.14 | NEUTRAL | −1.77 | −0.07 | 0.89 | 0 | H: 0.72 |
| L:PHE11>PRO.L:VAL26>TRP | | | | | | | L: 3.18 |
| H:LEU11>TRP.H:LEU28>ARG. | −0.06 | NEUTRAL | −5.26 | 2.92 | 1.26 | 0 | H: 0.72 |
| L:PHE11>ARG.L:VAL26>TRP | | | | | | | L: 3.37 |
| H:LEU11>TRP.H:LEU28>ARG. | 0.43 | NEUTRAL | −1.35 | 0.13 | 1.18 | 0 | H: 0.72 |
| L:PHE11>MET.L:VAL26>TRP | | | | | | | L: 2.97 |

### Example 6: Mutation Pair Development

For Pair 2, alanine/tryptophan single mutations were tested for each interface residue. IgG(-Fv) without VH and VL was constructed and expressed for Ala and Trp screening. Mutation list is listed as below.

| Name | Description | Cκ | CH1 |
|---|---|---|---|
| Cκ/CH1_001 | Cκ/CH1 | WT | WT |
| Cκ/CH1_002 | Cκ_L28Y_S69W/CH1_H51A_F53G | L28Y_S69W | H51A_F53G |
| Cκ/CH1_003 | Cκ/CH1_H51A_F53G | WT | H51A_F53G |
| Cκ/CH1_004 | Cκ/CH1_D31K_F53T_V68F | WT | D31K_F53T_V68F |
| Cκ/CH1_005 | Cκ/CH1_F9A_F53A | WT | F9A_F53A |
| Cκ/CH1_006 | Cκ_F9G_F11A_K100A/CH1 | F9G_F11A_K100A | WT |
| Cκ/CH1_007 | Cκ_F11A/CH1 | F11A | WT |
| Cκ/CH1_008 | Cκ_F9A_F11A/CH1 | F9A_F11A | WT |
| Cκ/CH1_009 | Cκ_F11A_K100A/CH1 | F11A_K100A | WT |
| Cκ/CH1_010 | Cκ_F9A_K100A/CH1 | F9A_K100A | WT |
| Cκ/CH1_011 | Cκ/CH1_F9A_L11A | WT | F9A_L11A |
| Cκ/CH1_012 | Cκ/CH1_L11A_F53A | WT | L11A_F53A |
| Cκ/CH1_013 | Cκ/CH1_F9A | WT | F9A |
| Cκ/CH1_014 | Cκ/CH1_L11A | WT | WA |
| Cκ/CH1_015 | Cκ_F9A/CH1 | F9A | WT |
| Cκ/CH1_016 | Cκ_F9A_F11M/CH1 | F9A_F11M | WT |
| Cκ/CH1_017 | Cκ/CH1_A24F | WT | A24F |
| Cκ/CH1_018 | Cκ/CH1_A24L | WT | A24L |
| Cκ/CH1_019 | Cκ_F9A_F11A/CH1_A24F | F9A_F11A | A24F |
| Cκ/CH1_020 | Cκ_F9A_F11A/CH1_A24L | F9A_F11A | A24L |
| Cκ/CH1_021 | Cκ_F9A_F11M/CH1_A24F | F9A_F11M | A24F |
| Cκ/CH1_022 | Cκ_F9A_F11M/CH1_A24L | F9A_F11M | A24L |
| Cκ/CH1_023 | Cκ_V26A/CH1 | V26A | WT |
| Cκ/CH1_024 | Cκ_V26A_F11A/CH1 | V26A_F11A | WT |
| Cκ/CH1_025 | Cκ/CH1_L11F_L28G | WT | L11F_L28G |
| Cκ/CH1_026 | Cκ_V26A/CH1_L11F_L28G | V26A | L11F_L28G |
| Cκ/CH1_027 | Cκ_V26A_F11A/CH1_L11F_L28G | V26A_F11A | L11F_L28G |
| Cκ/CH1_028 | Cκ/CH1_A24W | WT | A24W |
| Cκ/CH1_029 | Cκ/CH1_L11F | WT | L11F |
| Cκ/CH1_030 | Cκ/CH1_L11W | WT | L11W |
| Cκ/CH1_031 | Cκ_F9A_F11A/CH1_L11F_A24F | F9A_F11A | L11F_A24F |
| Cκ/CH1_032 | Cκ_V26W/CH1 | V26W | WT |

### Example 7: Alteration of Salt Bridges

The interface interaction analysis for Ck and CH1 in example 1 has shown that the common salt bridge between CH1 and Ck of 1F8, 1CZ8, 1L7I and 4NYL is below:

| CH1 | | | Cκ | | | |
|---|---|---|---|---|---|---|
| | | Atom | | | Atom | Distance |
| Position | Residue | Name | Position | Residue | Name | (Å) |
| 96 | LYS | NZ | 16 | GLU | OE2 | 2.7-3.4 |

There is one more salt bridge in 1F8 and 1CZ8:

| CH1 | | | Cκ | | | |
|---|---|---|---|---|---|---|
| | | Atom | | | Atom | Distance |
| Position | Residue | Name | Position | Residue | Name | (Å) |
| 101 | LYS | NZ | 15 | ASP | OD2 | 2.7-3.8 |

Therefore, this example focused on CH1 and Ck of 1F8 with two salt bridges and utilized the Discovery Studio to design new salt bridge pairs within CH1 and Ck that disfavor the binding of mutated CH1 or Ck to their WT counterpart and rebuild the binding between the mutated CH and Ck with a new salt bridge.

The design on the salt bridge CH1_LYS96 and Ck_GLU16. As shown in the below table, two pairs showed to stabilize CH1^{mut} and Ck^{mut} with new salt bridge:

CH1: LYS96>ASP mutation and Ck: GLU16>ARG mutation;
CH1: LYS96>GLU mutation and Ck: GLU16>ARG mutation;

| | Mutation | | | Electro | | Non- | Single |
|---|---|---|---|---|---|---|---|
| Mutation | Energy | Effect | VDW | static | Entropy | polar | Mutation |
| H:LYS96>ASP.L: | −1.06 | Stabilizing | −2.86 | 1.02 | −0.16 | 0 | 1.02 1.32 |
| GLU16>ARG | | | | | | | |
| H:LYS96>GLU.L: | −0.52 | Stabilizing | −2.38 | 0.98 | 0.21 | 0 | 1.28 1.32 |
| GLU16>ARG | | | | | | | |
| H:LYS96>ASP.L:G | −0.41 | Neutral | −2.06 | 1.08 | 0.09 | 0 | 1.02 1.84 |
| LU16>LYS | | | | | | | |
| H:LYS596>GLU.L: | 0.12 | Neutral | −0.24 | 1.74 | −0.72 | 0 | 1.28 0.79 |
| GLU16>HIS | | | | | | | |
| H:LYS96>GLU.L: | 0.26 | Neutral | −1.6 | 1.43 | 0.39 | 0 | 1.28 1.84 |
| GLU16>LYS | | | | | | | |

Discovery Studio was further used to find new salt bridge that could be in synergy with new Cκ_V26W and CH1_L11W to disfavor the binding of mutated CH1 or Ck to their WT counterpart and rebuild the binding between the mutated CH and Cκ. As shown in the below tables: three pairs showed to stabilize CH1^{mut} and Ck^{mut} with in synergy with Cκ V26W and CH1_L11W:

CH1: LEU11>TRP; LYS96>GLU mutation and Ck: GLU16>LYS; VAL26>TRP mutation
CH1: LEU11>TRP; LYS96>GLU mutation and Ck: GLU16>ARG; VAL26>TRP mutation
CH1: LEU11>TRP; LYS101>GLU mutation and Ck: ASP15>LYS; VAL26>TRP mutation

TABLE 5
Mutations in CH1_K96/Cκ_E16

| | Mutation | | | Electro- | | Non- | Double |
|---|---|---|---|---|---|---|---|
| Mutation | Energy | Effect | VDW | static | Entropy | polar | mutation |
| H:LEU11>TRP.H:LYS96>GLU. | −1.06 | Stabilizing | −4.09 | 1.61 | 0.2 | 0 | −0.34 |
| L:GLU16>LYS.L:VAL26>TRP | | | | | | | 3.39 |
| H:LEU11>TRP.H:LYS96>GLU. | −0.57 | Stabilizing | −1.6 | 1.94 | −0.84 | 0 | −0.34 |
| L:GLU16>ARG.L:VAL26>TRP | | | | | | | 2.03 |
| H:LEU11>TRP.H:LYS96>GLU. | −0.5 | Neutral | −0.79 | 2.11 | −1.32 | 0 | −0.34 |
| L:GLU16>HIS.L:VAL26>TRP | | | | | | | 1.11 |

TABLE 6
Mutations in CH1_K101/Cκ_D15

| | Mutation | | | Electro- | | non- | Double |
|---|---|---|---|---|---|---|---|
| mutation | Energy | Effect | VDW | static | Entropy | polar | mutation |
| H:LEU11>TRP.H:LYS101>GLU | −0.65 | Stabilizing | −2.28 | 2.18 | −0.68 | 0 | 0.58 |
| L:ASP15>LYS.L:VAL26>TRP | | | | | | | 1.57 |
| H:LEU11>TRP.H:LYS101>GLU. | −0.06 | Neutral | −1.66 | 2.08 | −0.31 | 0 | 0.58 |
| L:ASP15>HIS.L:VAL26>TRP | | | | | | | 1.66 |
| H:LEU11>TRP.H:LYS101>ASP. | 0.27 | Neutral | 1.33 | 1.91 | −1.53 | 0 | 0.62 |
| L:ASP15>LYS.L:VAL26>TRP | | | | | | | 1.57 |
| H:LEU11>TRP.H:LYS101>ASP. | 0.44 | Neutral | 1.46 | 1.96 | −1.44 | 0 | 0.62 |
| L:ASP15>ARG.L:VAL26>TRP | | | | | | | 1 |

### Example 8: Testing of Altered Salt Bridges

Plasmids containing polynucleotides encoding CH1-CH2-CH3 or Cκ were constructed. Mutations were introduced in some of the domains as listed below.

Plasmids were transiently transfected into 293F cells for protein expression. The proteins were purified by protein A columns and anti-FLAG affinity gel, and the purified proteins were analyzed by SDS-PAGE (5 μg per lane). As protein A binds to the heavy chain only, the density of the light chains indicated strength of binding between the heavy chain and the light chain.

In the first batch, 13 antibodies were tested. The mutations included in these antibodies are listed in Table 7.

TABLE 7
Test antibodies with mutations

| No. | Protein name | CH1-CH2CH3 | Cκ |
|---|---|---|---|
| 1 | Cκ/CH1_001 | WT | WT |
| 2 | Cκ/CH1_200 | WT | E16R |
| 3 | Cκ/CH1_201 | K96D | WT |
| 4 | Cκ/CH1_202 | K96E | WT |
| 5 | Cκ/CH1_203 | K96D | E16R |
| 6 | Cκ/CH1_204 | K96E | E16R |
| 7 | Cκ/CH1_107 | L11W | V26W |
| 8 | Cκ/CH1_205 | L11W; K96E | WT |
| 9 | Cκ/CH1_206 | WT | E16K; V26W |
| 10 | Cκ/CH1_207 | L11W; K96E | E16K; V26W |
| 11 | Cκ/CH1_208 | L11W; K96D | WT |
| 12 | Cκ/CH1_209 | WT | V26W; E16R |
| 13 | Cκ/CH1_210 | L11W; K96D | V26W; E16R |

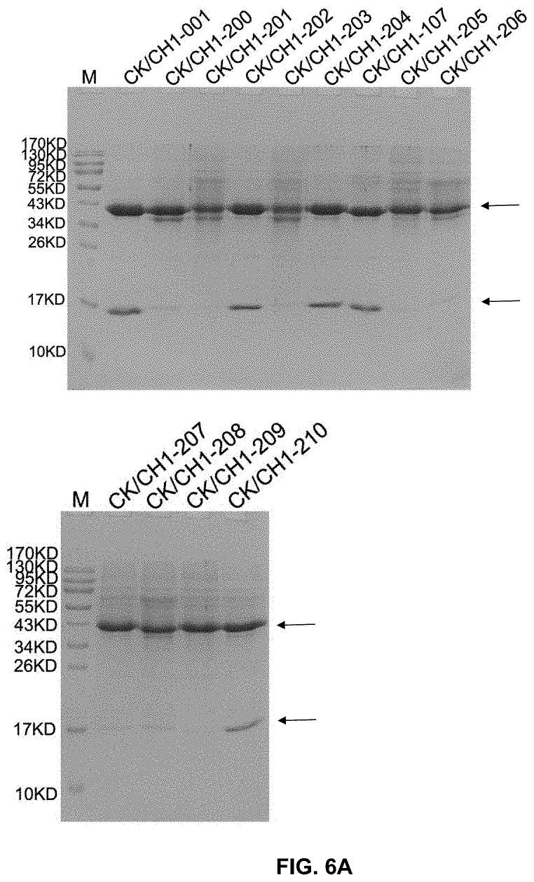

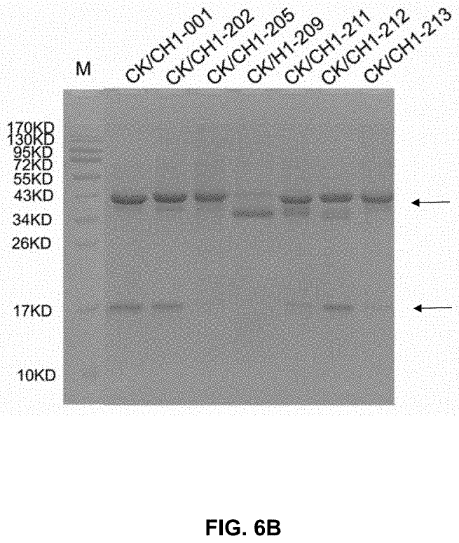

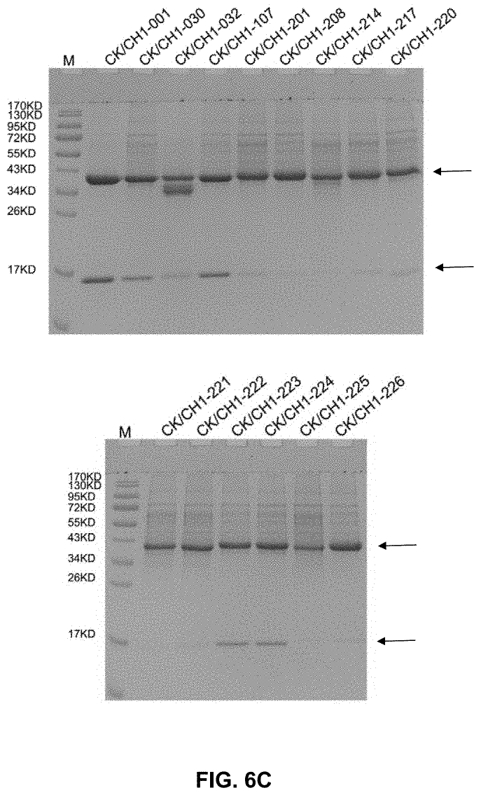

The results are shown in FIG. 6A. Good bindings were observed for Cκ/CH1_001 (wild-type) and Cκ/CH1_107 (L11W in CH1 and V26W in Cκ). Cκ/CH1_203 included a positive-to-negative and negative-to-positive mutation pair that disrupted the wild-type salt bridge (K96-E16). The binding in Cκ/CH1_210 (L11W and K96D in CH1 and V26W and E16R in Cκ) was markedly stronger than that between K96D and E16R. Each of the mutant chains, by contrast, more clearly failed to bind to the wild-type counterpart (see, Cκ/CH1_208 and Cκ/CH1_209).

The mutant chains in Cc/CH1_207, CH1 with L11W and K96E, and Cκ with E16K and V26W also exhibited more binding within mutants than their wild-type counterparts (see, Cκ/CH1_205 and Cκ/CH1_206).

In the second batch, seven antibodies were tested. The mutations included in these antibodies are listed in Table 8.

TABLE 8
Test antibodies with mutations

| | Protein name | CH1-CH2CH3 | Ck |
|---|---|---|---|
| 1 | Cκ/CH1_001 | Wt | Wt |
| 2 | Cκ/CH1_211 | Wt | E16K |
| 3 | Cκ/CH1_202 | K96E | Wt |
| 4 | Cκ/CH1_212 | K96E | E16K |
| 5 | Cκ/CH1_205 | L11W, K96E | Wt |
| 6 | Cκ/CH1_209 | Wt | E16R,V26W |
| 7 | Cκ/CH1_213 | L11W, K96E | E16R,V26W |

The results are shown in FIG. 6B. The mutant chains in Cκ/CH1_213, CH1 with L11W and K96E, and Cκ with E16R and V26W exhibited more binding within mutants than their wild-type counterparts (see, Cκ/CH1_205 and Cκ/CH1_206).

In the third batch, fifteen antibodies were tested. The mutations included in these antibodies are listed in Table 9.

TABLE 9
Test antibodies with mutations

| No. | Protein name | CH1-CH2CH3 | Cκ |
|---|---|---|---|
| 1 | Cκ/CH1_001 | WT | WT |
| 2 | Cκ/CH1_030 | L11W | WT |
| 3 | Cκ/CH1_032 | WT | V26W |
| 4 | Cκ/CH1_107 | L11W | V26W |
| 5 | Cκ/CH1_201 | K96D | WT |
| 6 | Cκ/CH1_214 | WT | C16R, Q17A |
| 7 | Cκ/CH1_217 | K96D | E16R, Q17A |
| 8 | Cκ/CH1_208 | L11W, K96D | WT |
| 9 | Cκ/CH1_225 | WT | E16R, Q17A, V26W |
| 10 | Cκ/CH1_226 | L11W, K96D | E16R, Q17A, V26W |
| 11 | Cκ/CH1_221 | WT | D15K, V26W |
| 12 | Cκ/CH1_222 | WT | D15H, V26W |
| 13 | Cκ/CH1_220 | L11W, K101E | WT |
| 14 | Cκ/CH1_223 | L11W, K101E | D15K, V26W |
| 15 | Cκ/CH1_224 | L11W, K101E | D15H, V26W |

As shown in FIG. 6C, the reestablished salt bridges in C/CH1_223 (K101E-D15K) and Cκ/CH1_224 (K101E-D15H) resulted in strong interactions between the mutated heavy and light chains, and each of them individually was more clearly unable to bind the wild-type counterpart as compared with Cκ/CH1_107 (L11W in CH1 and V26W in Cκ). The strong binding between the mutants, as shown in the figure, is also based on the hydrophobic interaction between L11W and V26W. In other words, the synergy between the hydrophobic interaction and the new salt bridge brings about strong binding and high specificity which will be useful for design of multi-specific antibodies.

### Example 9: Bi-Specific Antibody Construction

To further evaluate the effect of CH1/Ck mutations on light chain mismatch, we used IgG like heterodimer bi-specific format by using DE/EE mutations in CH3 domain (J. Biol. Chem. (2017) 292(35) 14706-14717). We constructed bi-specific antibodies by using the PDL1/CD73 pair.

The PDL1/CD73 pair design is described in the table below:

| | Protein | | | | | | | |
|---|---|---|---|---|---|---|---|---|
| | B5021 | | B5022 | | B5023 | | B5024 | |
| | CH3 | | | | | | | |
| | DE/KK | | DE/KK | | DE/KK | | DE/KK | |
| Fab | PDL1 | CD73 | PDL1 | CD73 | PDL1 | CD73 | PDL1 | CD73 |
| CH1 | K96D | L11W | K96D | WT | L11W/ | WT | L11W/ | WT |
| | | | | | K96D | | K96E | |
| Ck | E16K | V26W | E16K | WT | V26W/ | WT | V26W/ | WT |
| | | | | | E16R | | E16K | |

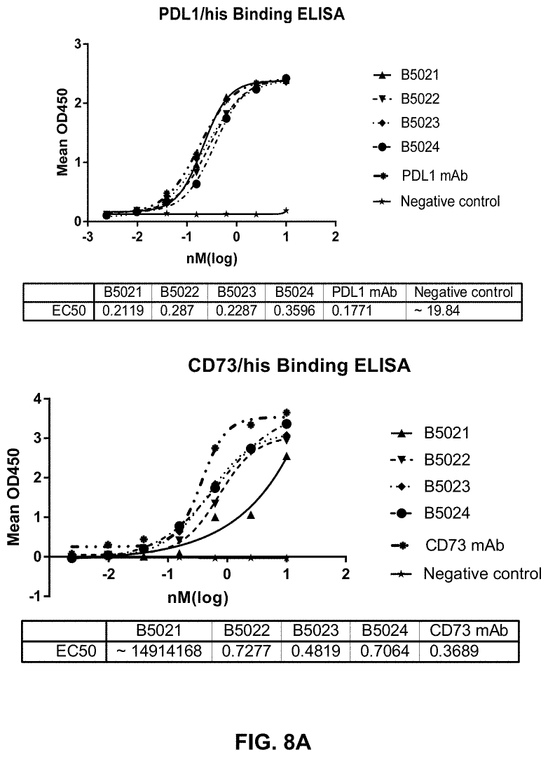

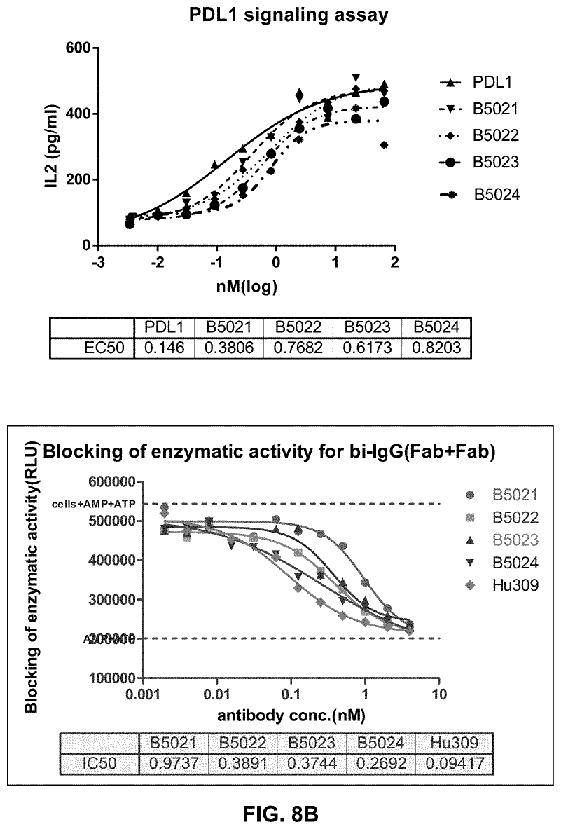

As shown in FIG. 8A, all the designed pairs didn't affect the PDL1 part binding by ELISA, while the binding potency of CD47 was impaired. B5024 Cκ/CH1_207 mutations (CH1:L11W/K96E; Cκ: E16KN26W) and B5023 Cκ/CH1_210 mutations (CH1:L11W/K96D; Cκ: E16RNV26W) can restore the CD73 part antigen binding by ELISA. In addition, the PDL1 singling assay and CD73 enzymatic activity assay showed similar pattern with ELISA binding (FIG. 8B). In this regard, all the PDL1 part showed similar PDL1 antagonism activity and only B5024 and B5023 showed potent CD73 antagonist activity. In this pair, the light chain of PDL1 significantly impaired the function of CD73 arm, while CD73 light chain has little effect on PDL1 arm. Both Cκ/CH1_207 and Cκ/CH1_210 mutations can restore the function of CD73 and didn't affect the PDL1 arm, suggesting CH1/Ck mutations can prevent the light chain mismatch.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference

1. An antibody or antigen-binding fragment thereof, comprising a human CH1 fragment comprising a L11W substitution and a human Cκ fragment comprising a V26W substitution.

2. The antibody or antigen-binding fragment thereof of claim 1, wherein the CH1 fragment comprises substitutions L11W and K101E and the Cκ fragment comprises substitutions V26W and D15K/H.

3. The antibody or antigen-binding fragment thereof of claim 1, wherein the CH1 fragment comprises substitutions L11W and K96D and the Cκ fragment comprises substitutions V26W and E16R.

4. The antibody or antigen-binding fragment thereof of claim 1, wherein the CH1 fragment comprises substitutions L11W and K96E and the Cκ fragment comprises substitutions V26W and E16K.

5. The antibody or antigen-binding fragment thereof of claim 1, wherein the CH1 fragment comprises substitutions L11W and K96E and the Cκ fragment comprises substitutions V26W and E16R.

6. The antibody or antigen-binding fragment thereof of claim 1, further comprising a second human CH1 fragment that does not include the L11W substitution and a second human Cκ fragment that does not include the V26W substitution.

7. The antibody or antigen-binding fragment thereof of claim 6, wherein the second human CH1 and the second human Cκ fragments are wild-type.

8. The antibody or antigen-binding fragment thereof of claim 1, further comprising a heavy chain variable region, a light chain variable region, an Fc region, or the combination thereof.

9. The antibody or antigen-binding fragment thereof of claim 8, which is of class IgG.

10. The antibody or antigen-binding fragment thereof of claim 9, wherein the isotype is IgG1, IgG₂, IgG₃ or IgG₄.

11. An antibody or antigen-binding fragment thereof, comprising a human CH1 fragment to human Cκ fragment pair, wherein the CH1 and Cκ fragments comprise substitutions selected from the group consisting of:
(a) L11K and L28N in CH1, and V26W in Cκ;
(b) L11W in CH1, and F11W and V26G in Cκ;
(c) F9D in CH1, and Q17R or Q17K in Cκ; and
combinations thereof.

12. The antibody or antigen-binding fragment thereof of claim 11, wherein the CH1 and Cκ fragments further comprise substitutions selected from the group consisting of (a) K101E in CH1 and D15K/H in Cκ, (b) K96D in CH1 and E16R in Cκ, (c) K96E in CH1 and E16K in Cκ and (d) K96E in CH1 and E16R in Cκ.

13. An antibody or antigen-binding fragment thereof, comprising a human CH1 fragment comprising an amino acid substitution at position Leu11, and a human Cκ fragment comprising an amino acid substitution at position V26 and/or F11, wherein the substituted amino acids interact with each other when the CH1 fragment pairs with the Cκ fragment.

14. The antibody or antigen-binding fragment thereof of claim 13, wherein the human CH1 fragment does not interact with a wild-type human Cκ domain and the human Cκ domain does not interact with a wild-type human CH1 fragment.

15. The antibody or antigen-binding fragment thereof of claim 13, wherein the amino acid substitutions are selected from Table 1.

16-18. (canceled)

19. The antibody or antigen-binding fragment thereof of claim 11, further comprising a heavy chain variable region, a light chain variable region, an Fc region, or the combination thereof.

20-21. (canceled)

22. A composition comprising the antibody or antigen-binding fragment thereof of claim 1 and a pharmaceutically acceptable carrier.

23. An isolated cell comprising one or more polynucleotide encoding the antibody or antigen-binding fragment thereof of claim 1.
